(19) United States
(12) Patent Application Publication
(10) Pub. No.: US 2015/0352142 A1
(43) Pub. Date: Dec. 10, 2015

(54) STABILIZED COMPOSITIONS COMPRISING HYALURONIC ACID

(71) Applicant: Carbylan Therapeutics, Inc., Palo Alto, CA (US)

(72) Inventors: David M. Gravett, Mountain View, CA (US); Pingren He, Sunnyvale, CA (US)

(21) Appl. No.: 14/760,447

(22) PCT Filed: Jan. 10, 2014
(86) PCT No.: PCT/US2014/011160

§ 371 Date: Jul. 10, 2015

Related U.S. Application Data
(60) Provisional application No. 61/751,811, filed on Jan. 11, 2013.

(51) Int. Cl. A61K 31/728; A61K 31/58; A61K 47/36; A61K 47/10; A61K 9/06; C08L 5/08; A61K 47/26

(52) U.S. Cl. CPC A61K 31/728; C08L 5/08; A61K 31/58; A61K 47/26; A61K 47/10; A61K 9/06; A61K 47/36

(57) ABSTRACT

Provided herein are hyaluronic-acid based compositions comprising a hyaluronic acid-based polymer and a corticosteroid in the presence of a stabilizing amount of one or more stabilizing additives and related methods. The resulting compositions substantially retain their viscoelastic and related rheolotical properties under conditions of storage, processing, and use.

### CROSS-REFERENCE TO RELATED APPLICATIONS

This application claims the benefit of priority to U.S. Provisional Patent Application No. 61/751,811, filed Jan. 11, 2013, the content of which is incorporated herein by reference in its entirety.

### FIELD

The disclosure relates generally to compositions comprising hyaluronic acid and/or hyaluronic acid-based polymers, and related methods of making, using, and stabilizing such compositions. The resulting biocompatible compositions, e.g., pharmaceutical compositions, can be used, for example, for localized delivery of bioactive agents, and for treating various medical conditions. The resulting compositions, in general, better retain their viscosity over time when compared to compositions absent a stabilizing amount of a stabilizing additive, to be described in greater detail below.

### BACKGROUND

Hyaluronic acid is a naturally-occurring, anionic, non-sulfated glycosaminoglycan that is distributed widely throughout connective, epithelial, and neural tissues. The average 70 kg (154 lbs) person possesses roughly 15 grams of hyaluronic acid in his/her body, one-third of which is turned over (degraded and synthesised) every day (Stern R. Eur J Cell Biol 83 (7): 317-25, (2004)). Since hyaluronic acid is found naturally in many tissues of the body and is therefore biocompatible, it is believed to be well suited for biomedical applications. Unfortunately, hyaluronic acid, when administered and used as a therapeutic in its naturally occurring form, is typically rapidly cleared from the body, making frequent administration necessary. Thus, formulations of hyaluronic acid which maintain the benefits of unaltered hyaluronic acid such as good biocompatibility, but which overcome the problem of rapid clearance are highly desirable. Such formulations should ideally have good cytocompatibility, beneficial chemical, rheological and other properties, good stability, and possess an ease of administration.

Chemically modified forms of hyaluronic acid have been previously described. See, e.g., J. W. Kuo, et al., Materials of Biological Origins—Materials, Analysis, and Implant Uses, Comprehensive Biomaterials, Elsevier, 2010. Hyaluronic acid (also referred to as hyaluronan), derivatized forms thereof, related compositions and conjugates, can be used as injectable biomaterials, as well as in medical devices and implantable materials. Applications include delivery of therapeutic molecules to a localized site, use as adhesives or sealants, in tissue engineering, as viscosupplements, and in wound healing, among others. Hyaluronic-based materials can be prepared in several different forms—viscoelastic solutions, soft or stiff hydrogels, non-woven meshes, sponges, and the like, and can be used in a range of preclinical and clinical settings. For many preclinical and clinical uses, it is important to maintain the viscoelastic properties of such hyaluronic acid-based materials.

Thus, it would be highly advantageous to be able to provide hyaluronic-acid based compositions which substantially maintain their viscoelastic properties, i.e., exhibit good chemical and physical stability, e.g., under conditions of storage, processing, and use.

### SUMMARY

The current disclosure is based, at least in part, upon the Applicant's discovery that hyaluronic acid polymer-based compositions comprising a corticosteroid such as triamcinolone acetonide or others, exhibit a loss of viscosity over time that is significantly greater than that observed for the same HA polymer-based compositions absent the corticosteroid. Based upon the foregoing, the Applicants have identified certain materials effective to significantly stabilize such HA polymer-comprising compositions, such that the loss in viscosity over time is substantially diminished by the incorporation of such one or more stabilizing additives. Thus, provided herein are compositions which better retain their viscosity over time (i.e., exhibit a reduced loss in viscosity over time) when compared to comparable compositions absent the stabilizing additive. Also provided herein is a method for improving the storage stability of a hyaluronic acid-based composition comprising a corticosteroid by incorporating into such composition a stabilizing amount of an additive effective to result in a stabilization ratio (under conditions such as those to be described in greater detail below) of greater than 1, preferably greater than 1.2, more preferably greater than 1.3, where the stabilization ratio is a measure of diminished loss in viscoscity over time for the subject composition comprising the stabilizing additive in comparison to the same composition absent the stabilizing additive. (A stabilization ratio greater than 1 represents an enhancement of percent retention of viscosity at a given time point and under certain storage conditions over the control composition absent additive).

Additional embodiments of the compositions, methods, uses and the like will be apparent from the following description, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description, particularly when considered in conjunction with the accompanying examples and drawings.

### BRIEF DESCRIPTION OF THE FIGURES

This application is accompanied by no figures.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

All publications, patents and patent applications cited herein, whether supra or infra, are hereby incorporated by reference in their entirety, unless otherwise indicated. In an instance in which the same term is defined both in a publication, patent, or patent application incorporated herein by reference and in the present disclosure, the definition in the present disclosure represents the controlling definition. For publications, patents, and patent applications referenced for their description of a particular type of compound, chemistry, etc., portions pertaining to such compounds, chemistry, etc. are those portions of the document which are incorporated herein by reference.

### Definitions

It must be noted that, as used in this specification, the singular forms “a,” “an,” and “the” include plural referents unless the context clearly dictates otherwise. Thus, for example, reference to a “polymer” includes a single polymer as well as two or more of the same or different polymers.

Unless specifically noted otherwise, definitions of the terms herein are standard definitions used in the arts of organic synthesis, and polymer and pharmaceutical science.

In describing and claiming the present invention, the following terminology will be used in accordance with the definitions described below.

A “biocompatible polymer” is a polymer having degradation products that are compatible with living tissue, or that may have beneficial biological properties. The biocompatible polymer may be biocompatible in itself, and/or may be synergistically biocompatible when employed in conjunction with a biologically active agent.

The term “hyaluronic acid polymer” refers to a polymer comprising repeat disaccharide subunits of hyaluronan, where the repeat units may be derivatized at one or more positions of the D-glucuronic acid and/or the D-N-acetylglucosamine unit of the disaccharide repeat subunit. A hyaluronic acid polymer or hyaluronic acid-based polymer is meant to encompass hyaluronic acid (also referred to as hyaluronan), derivatized hyaluronic acid, salts forms, and hyaluronic acid linker complexes.

The term, “hyaluronic acid”, is meant to refer to unmodified or non-derivatized hyaluronic acid.

The terms “hyaluronic acid derivative” or “derivatized hyaluronic acid” or “modified hyaluronic acid” or “substituted hyaluronic acid” refers to hyaluronic acid that has been chemically modified.

The term “reactive” refers to a functional group (e.g., present in a polymer) that reacts readily or at a practical rate under conventional conditions of organic synthesis. This is in contrast to those groups that either do not react or require strong catalysts or impractical reaction conditions in order to react (i.e., a “nonreactive” or “inert” group).

“Molecular mass” or molecular weight, as used herein, in the context of a water-soluble polymer such as hyaluronic acid, refers to the nominal average molecular mass of a polymer determined by multi angle light scattering. Molecular weight can be expressed as either a number-average molecular weight or a weight-average molecular weight. Unless otherwise indicated, all references to molecular weight herein refer to the number-average molecular weight. In the absence of a molecular weight value, a polymer may also be characterized by its intrinsic or inherent viscosity, which is a viscometric method for measuring molecular weight.

The term “hydrogel” refers to a water-containing three dimensional hydrophilic polymer network or gel in which the water is the continuous phase, for example, in which the water content is greater than 50% (w/w).

By “gelation” is meant the formation of a material into a gelled state.

A “sterile” composition is one that is free of viable microbes as determined using the USP sterility test. See “The United States Pharmacopeia”, 30th Revision, The United States Pharmacopeial Convention: 2008.

The term “drug,” or “pharmaceutically active agent” or “bioactive agent,” or “active agent” as used interchangeably, means any organic or inorganic compound or substance having bioactivity and adapted or used for therapeutic purposes. Proteins, hormones, anti-cancer agents, small molecule chemical compounds and mimetics, oligonucleotides, DNA, RNA and gene therapies are included under the broader definition of “drug”. As used herein, reference to a drug such as a corticosteroid, as well as reference to other chemical compounds herein, is meant to include the compound in any of its pharmaceutically acceptable forms, including isomers such as diastereomers and enantiomers, salts, solvates, and polymorphs, particular crystalline forms, as well as racemic mixtures and pure isomers of the compounds described herein, where applicable.

A “water insoluble drug” or “poorly water soluble drug” is one having an aqueous solubility below 10 mg/mL.

The terms “effective amount” or “pharmaceutically effective amount” or “therapeutically effective amount” of a composition (or hydrogel or polymer), as provided herein, refer to a non-toxic but sufficient amount of the composition to provide the desired response, such as preventing, diminishing, or eliminating pain in a subject. The exact amount required will vary from subject to subject, depending on the species, age, and general condition of the subject, the severity of the condition being treated, the particular drug or drugs employed, specifics of the composition, mode of administration, and the like. An appropriate “effective” amount in any individual case may be determined by one of ordinary skill in the art using routine experimentation.

“Optional” or “optionally” means that the subsequently described circumstance may or may not occur, so that the description includes instances where the circumstance occurs and instances where it does not.

The term “substantially” in reference to a certain feature or entity means to a significant degree or nearly completely (i.e. to a degree of 85% or greater) in reference to the feature or entity.

The term “about”, particularly in reference to a given quantity, is meant to encompass deviations of plus or minus five percent.

Additional definitions may also be found in the sections which follow.

### Overview

The present application is based, at least in part, on the Applicant's discovery of certain additives effective to stabilize compositions, typically aqueous compositions, comprising a hyaluronic acid-based polymer and a corticostertoid. As described previously, the Applicants recognized that aqueous compositions comprising a hyaluronic acid-based polymer and a corticostertoid exhibit a loss of viscosity over time that is significantly greater than that observed for the same HA polymer-based compositions absent the corticosteroid. Based upon the foregoing, the Applicants have identified certain materials effective to significantly stabilize such HA polymer-comprising compositions, such that the loss in viscosity over time is substantially diminished by the incorporation of such one or more stabilizing additives.

In the present context, by “stabilize” is meant to reduce the extent of loss of viscosity (although other measures can be used) observed for the hyaluronic acid polymer-corticosteroid comprising (HA-CORT) composition over time, e.g., when compared to the same hyaluronic acid polymer-corticosteroid comprising composition absent the additive. As can be seen from Examples 1-27, when exposed to accelerated storage stability conditions (i.e., accelerated temperatures over extended periods of time), HA-CORT compositions comprising stabilizing amounts of certain sugars (monosaccharides, disaccharides, polysaccharides), sugar alcohols, polyols, and polyol containing surfactants, exhibit significantly enhanced retention of viscosity in comparison to HA-CORT compositions absent such stabilizing additive as conveniently indicated by their stabilization ratios. The stabilization ratio is the ratio of the percent retention of HA viscosity for the HA-CORT stabilizing additive-containing composition (HA-CORT-SA) to the percent retention of HA viscosity of the same HA-CORT composition (absent additive) measured at a certain time point and under certain storage conditions. In this way, by normalizing the data, the effect of the additives can be readily compared. In the compositions prepared, data for the hyaluronic acid polymer-only compositions demonstrated the destabilizing effect of the corticosteroid on the viscosity of the HA, as evidenced by its diminishment. See, for example, Example 1A. In cases with materials effective as stabilizing additives, the percent retention of viscosity over time was significant. For example, particularly useful compositions are those that exhibit a reduction in viscosity of no more than about 50% when compared to their initial viscosity values (at time zero) when stored at 60° C. for a period of 6 days. Materials suitable as stabilizing additives will generally exhibit a stabilization ratio for the corresponding composition at day 6 when stored at 60° C. of at least 1.2, and preferably of at least 1.3. As can be seen from the examples, effective additives resulted in stabilization ratios ranging from about 1.2 to about 2.6 or greater under the above conditions. (The choice of accelerated storage conditions/shelf life is arbitrary and simply provides a convenient measure of assessing materials suitable for use as additives in the subject compositions). As can be seen in the supporting examples, HA-based compositions were examined under a variety of temperatures (40° C., 45° C., 55° C., 60° C., 80° C., etc.), humidities (ambient and increased humidity), and storage periods (hours such as 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year or greater)). As can be seen in the Examples, more pronounced effects are generally seen for samples stored under more extreme conditions, such as high temperatures (see for instance, Example 13, carried out at 80° C. (observed stabilization ratios ranging from about 5 to about 7), or for extended periods of time (see, e.g., Example 22 in which the stabilized composition possessed a stabilization ratio of 7.47).

### Compositions

### HA-Based Components

The hyaluronic acid polymer used in the subject compositions and methods may be derivatized or underivatized, and may or may not be crosslinked. Moreover, the subject compositions may comprise more than one type of hyaluronic acid such as chemically modified hyaluronic acid (which itself may or may not be crosslinked), crosslinked hyaluronic acid, unmodified hyaluronic acid, or combinations of the foregoing. More specifically, the hyaluronic acid polymer component(s) can be in a fluid form or in a gel form or a combination of both. In one preferred embodiment, the hyaluronic acid polymer comprises a modified hyaluronic acid gel suspended in an aqueous solution of unmodified hyaluronic acid.

One exemplary modified hyaluronic acid is hyaluronic acid derivatized by reaction of its hydroxyl groups with divinyl sulfone and then lightly crosslinked with a crosslinker such as PEG-dithiol. In a preferred embodiment, the hyaluronic acid is modified to a degree of 10% or less by reaction with divinyl sulfone to transform 10% or less of its hydroxyl groups to (2-(vinylsulfonyl)ethoxy) groups. In particular, the hyaluronic acid may possess a degree of conversion of hydroxyl groups to (2-(vinylsulfonyl)ethoxy) groups selected from the following: 1%, 2%, 3%, 4%, 5%, 6%, 7%, 8%, 9%, and 10%. Alternatively, the hyaluronic acid may possess a degree of conversion of hydroxyl groups falling within a range between any two of the foregoing percentages: e.g., from 1-10%, 2-10%, 3-10%, 4-10%, and so forth for each and every combination of integers provided, e.g., from 2-7%, from 2-6%, from 3-8%, from 3-7%, and so forth. In yet a more specific embodiment, the hyaluronic acid has a degree of conversion of hydroxyl groups to (2-(vinylsulfonyl)ethoxy) groups of about 4-5% per disaccharide repeat unit. The degree of substitution/modification of the parent polymer can be determined by any of a number of suitable methods, e.g., NMR, UV, or IR analysis, or elemental analysis. The resulting activated hyaluronic acid is referred to generally as (2-(vinylsulfonyl)ethoxy)hyaluronic acid or VS-HA and is described in U.S. Pat. No. 7,829,118.

Examples of crosslinkers suitable for reacting with a hyaluronic acid polymer such as VS-HA include thiol-containing crosslinkers comprising two or more thiol groups. Such thiol groups will react with a vinyl sulfone such as within a vinyl-sulfone derivatized hyaluronic acid. Illustrative thiol cross linkers include PEG-dithiol (HS-PEG-SH), 3-arm PEG-tri-thiol (glycerine core), 4-arm PEG-tetrathiol (pentaerythritol core), or 8-arm PEG-octa-thiol (hexaglycerine core). A preferred crosslinker is PEG-dithiol. The molecular weight of the crosslinker is typically less than that of the modified hyaluronic acid. Generally, the molecular weight of the crosslinker ranges from about 200 to about 20,000 daltons. Exemplary molecular weights for a crosslinker such as PEG dithiol, or any of the other suitable crosslinkers, include about 3350, 3400, and 5000 daltons, among others. See, for example, Examples 28-33 herein. Example 28 describes the preparation of an exemplary vinyl sulfone derivatized hyaluronic acid having a level of vinyl sulfone modification of approximately 4%. Example 29 describes the preparation of a gel by reaction of vinyl sulfone derivatized hyaluronic acid having a low level of modification with the crosslinker, PEG-dithiol, having a molecular weight of 3400. Example 30 describes the preparation of a HA-VS/PEG-dithiol gel slurry in saline, while Example 31 describes the preparation of a HA-VS/PEG-dithiol gel slurry in a solution of hyaluronic acid. All of these formulations provide exemplary hyaluronic acid polymer components, e.g., aqueous solutions, gels, and combinations thereof, suitable for incorporation of a stabilizing additive.

The hyaluronic acid-based polymer may also comprise hydrazide-reactive groups and/or aminooxy-reactive groups as described in PCT/US/2004/040726 (WO 2005/056608), relevant portions of the disclosure related to the derivatization of HA and the resulting polymers themselves being incorporated herein by reference in their entireties.

Alternatively, the HA may be thiol-derivatized, such as a thiol-derivatized hyaluronic acid. Exemplary thiol-derivatized hyaluronic acid polymers include those described in U.S. Pat. Nos. 7,981,871; 6,884,788; 6,620,927; 6,548,081; 6,537,979; 6,013,679; 5,502,081; and 5,356,883, relevant portions of which related to such thiol-derivatized polymers being incorporated herein by reference in their entireties.

Additional examples of hyaluronic acid polymers include cysteine-derivatized hyaluronic acid, including but not limited to those polymers disclosed in “Controlled Release from Glycosaminoglycan Drug Complexes” R. V. Sparer et al., Chapter 6, pages 107-119, in T. J. Roseman et al., CONTROLLED RELEASE DELIVERY SYSTEMS, Marcel Dekker, Inc., New York (1983).

Examples of additional preferred polymers include hyaluronic acid derivatized by a pendent thiol group linked to an N-acyl urea group via a hydrocarbyl, aryl, substituted-hydrocarbyl, or substituted aryl group. Illustrative polymers for use in the compositions and methods provided herein include Carbylan™-S (described in detail in International Patent Publication No. WO 2005/056608).

Additionally, the hyaluronic acid or derivatized hyaluronic acid may be covalently attached to a reactive linker and/or crosslinked with a difunctional or multi-functional acrylate, allyl or methacrylate compound. Representative linkers for modification of hyaluronic acid include, but are not limited to, poly (ethylene glycol)-diacrylate (PEGDA), poly (ethylene glycol)-dimethacrylate (PEGDM), poly (ethylene glycol)-diacrylamide (PEGDAA) and poly (ethylene glycol) -dimethacrylamide (PEGDMA), and derivatives thereof. The PEG-moieties of the foregoing linkers may be oliogomeric or polymeric, for example, comprising from 2 to 100 or more subunits. Additional linkers suitable for modification/functionalization of a polymer such as hyaluronic acid include but are not limited to dextran acrylate, dextran methacrylate, dextran glycidyl methacrylate, methacrylate functionalized hyaluronic acid, acrylate functionalized hyaluronic acid, glycerol dimethacrylate, glycerol 1,3-diglycerolate diacrylate, sorbitol acrylate and derivatives thereof.

The hyaluronic acid or hyaluronic-based polymer will typically possess an average molecular weight in the range of about 700 to 3,000,000 daltons. Illustrative molecular weight ranges are from about 1,000 to 2,000,000 daltons, or from about 5,000 to 1,000,000 daltons. Additional suitable molecular weight ranges include from about 50,000 daltons to about 1,000,000 daltons, or from about 100,000 daltons to about 1,200,000 daltons, or from about 90,000 daltons to about 300,000 daltons. Molecular weights of hyaluronic acid are generally average molecular mass values, which can be determined, for example, by multi-angle laser light scattering exlusion chromatography (MALLS-SEC). Depending upon its source, hyaluronic acid may have a polydispersity (M_{w}/Mₙ) of up to around 3, or more preferably, up to around 2. Generally, the hyaluronic acid will have a rather narrow molecular weight distribution, with values less than about 2.5, more preferably less than about 2. Exemplary polydispersities of hyaluronic acid polymers range from about 1.02 to about 2.5, where the starting hyaluronic acid may possess a polydispersity of about 1.02, 1.05, 1.1, 1.2, 1.3, 1.3, 1.5, 1.6, 1.7, 1.8, 1.9, 2,0, 2.1, 2.2, 2.3, 2.4 or 2.5, or even greater. Alternatively, the hyaluronic acid polymer may have a viscosity, typically in centipoise, at a specific concentration in water, that corresponds to any one or more of the average molecular weight ranges provided above.

As described above, the hyaluronic acid component may comprise crosslinked HA-based hydrogel particles in an aqueous solution of hyaluronic acid. One such composition is described in Example 31. A preferred aqueous solution is a saline solution of hyaluronic acid, where exemplary aqueous solutions of hyaluronic acid added to the hydrogel have concentrations ranging from about 0.3% to about 4%, or from about 0.5% to about 2% by weight. One representative formulation comprises the following relative amounts of components: 4 mL of gel slurry ((2-(vinylsulfonyl)ethox)_{1-10%}hyaluronic acid/PEG-dithiol) with 2 mL of hyaluronic acid at a concentration of 20 mg/mL. A particularly preferred formulation comprises 4 mL of gel slurry ((2-(vinylsulfonyl)ethoxy)_{4%}hyaluronic acid/PEG-dithiol) with 2 mL of hyaluronic acid at a concentration of 20 mg/mL. Typically, the final hyaluronic acid content in the resulting swollen gel ranges from about 0.05 to 5 percent (0.5 mg/mL to 50 mg/mL). Preferably, the final hyaluronic acid content in the resulting swollen gel is from about 0.1 to 3 percent, or from about 0.1 to 1 percent, or from about 0.5-0.8%. Illustrative final hyaluronic acid content in the resulting swollen gel may, for example, correspond to any of the following percentages: 0.1, 0.2, 0.3, 0.4, 0.5, 0.6, 0.7, 0.8, 0.9, 1.0, 2.0, 3.0, 4.0, and 5.0. For example, representative relative amounts (weight ratios) of hyaluronic acid to crosslinked (e.g., (2-(vinylsulfonyl)ethoxy)_{1-10%}hyaluronic acid/PEG-dithiol) hydrogel particles in the resultant composition typically fall within a range from about 10:1, or from about 5:1, or from about 3:1, or from about 1:1.

Other exemplary HA-based compositions include those compositions referred to as SYNVISC® (a gel-like mixture comprising hylan A fluid, hylan B gel, and saline) HYLASTAN™, MONOVISC™, GEL-ONE® (see, e.g., U.S. Patent Publication No. 2012/01426229) or DUROLANE®, further comprising a corticosteroid. Additional hyaluronic acid-based compositions for use in combination with a corticosteroid and stabilizing additive as provided herein include those described in U.S. Pat. No. 4,713,448, U.S. Pat. No. 5,143,724, U.S. Pat. No. 5,827,937, U.S. Pat. No. 6,013,679, U.S. Pat. No. 5,356,883, U.S. Pat. No. 5,502,081, as well as U.S. Pat. Nos. 6,884,788, 6,548,081, 6,620,927.

In one or more embodiments, the hyaluronic acid polymer component provided herein is sterile. Typical methods of sterilization include heating, autoclaving, chemical treatment or filtration.

### Corticosteroid

The subject HA-polymer based compositions comprise a corticosteroid. As discussed previously, the Applicants recognized that aqueous hyaluronic acid polymer compositions containing a corticosteroid such as triamcinolone acetonide or others demonstrate a loss of viscosity over time that is significantly greater than that observed for the same compositions absent the corticosteroid. Thus, a need was recognized for providing hyaluronic acid polymer -corticosteroid compositions having a robust resistance to a reduction in viscosity over time.

Generally, the corticosteroid is selected from one or more of the following: hydrocortisone, hydrocortisone acetate, cortisone acetate, tixocortol pivalate, prednisolone, methylprednisolone, prednisone, triamcinolone, triamcinolone acetonide, triamcinolone benetonide, triamcinolone furetonide, triamcinolone hexacetonide, triamcinolone diacetate, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, beclomethasone dipropionate monohydrate, flunisolide, fluticasone propionate, mometasone furoate monohydrate, and fluticasone furoate.

In a preferred embodiment, the composition comprises triamcinolone acetonide. One preferred compound for use in a hydrogel formulation as provided herein is triamcinolone (11β,16α)-9-fluoro-11,16,17,21-tetrahydroxypregna-1,4-diene-3,20-dione), or a derivative thereof such as triamcinolone acetonide, or a pharmaceutically acceptable salt, ester, or solvate thereof. The structure of triamcinolone acetonide is shown below.

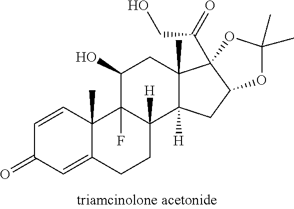

The corticosteroid will typically be admixed, suspended in, or entrapped within an HA-polymer based composition as provided herein. Alternatively, the corticosteroid may be in the form of a polymer conjugate, or, may be covalently attached, in a releasable fashion, to a component used to prepare the HA-based hydrogel.

Generally, the amount of corticosteroid contained in the composition is in a range of about 0.5-80 mg/mL. Alternatively, on a per weight basis, aqueous compositions as provided herein contain from about 0.01% by weight to about 20% by weight corticosteroid, depending on its potency. Illustrative amounts of corticosteroid (based on overall wet gel weight) are from about 10% to about 20% by weight, e.g., for a less potent corticosteroid, and from about 0.01% to about 10% by weight, or from about 0.01% to about 5%, or from about 0.01% to about 3%, or from about 0.1 to about 2% corticosteroid, or even from about 0.1 to about 1% corticosteroid, e.g., for a more potent bioactive agent such as triamcinolone acetonide.

### Stabilizing Additives

In attempts to stabilize hyaluronic-acid polymer—corticosteroid compositions as provided herein, the Applicants explored a number of different types of additives, many of which were ineffective at preventing or minimizing the reduction in viscosity observed. However, additives such as sugars, sugar alcohols, polyols and derivatized polyols as described herein were discovered to be particularly effective. Thus, stabilizing additives for incorporation into the instant HA-polymer based-corticosteroid compositions include certain sugars (monosaccharides, disaccharides, polysaccharides), sugar alcohols, polyols, and polyol-containing surfactants. Although such materials have been used to stabilize proteins from deactivation, generally during processing, such materials do not appear to have ever been reported or recognized for use in stabilizing HA-polymer-based materials against a loss of viscosity or other rheolotical properties when combined with a corticosteroid. In one embodiment, an aqueous, HA-based polymer composition as described herein is absent a therapeutic protein or peptide. In general, without being bound by theory, the stabilizing additives tend to possess one or more hydroxyalkyl groups such as the hydroxyl-lower alkyl groups hydroxymethyl (—CH₂—OH) or hydroxyethyl (—CH₂CH₂OH). Materials discovered to be suitable as stabilizing additives in the corticosteroid-containing compositions provided herein include polysaccharides such as sucrose and dextran-40 and the derivatized polysaccharide, carboxymethylcellulose; sugar alcohols such as mannitol and sorbitol; monosaccharides such as n-acetylglucosamine and dextrose; disaccharides such as trehalose, maltose, and lactose; polyols such as polyethylene glycol (PEG) and the derivatized polyol, TRITON™-X-100 (a non-ionic surfactant comprising an aromatic hydrocarbon moiety and a PEG chain, 4-(1,1,3,3-tetramethylbutyl)phenyl-polyethylene glycol), and glycerol (a simple polyol), as illustrated in the supporting Examples. Polyols such as PEG and derivatized polyols suitable for use possess a number of ethylene oxide repeat units from about 4 to about 500, or preferably from about 6 to about 100.

Examples 1, 2, 3, 4, 5, 6, 7, 8, 9, 10, 11, 12, 17, 18, 19, 20, 21, 22, 23, 24, 25, 26, 27 all demonstrate the superior performance of sucrose as a stabilizing additive in hyaluronic acid polymer-comprising solutions, and in hyaluronic acid polymer-solutions that additionally comprise a hyaluronic acid polymer-based gel. Thus, a preferred stabilizing additive is sucrose. The examples demonstrate that addition of sucrose is effective to impart a stabilization effect against a reduction in viscosity of 1.2 to at least 11.3 fold under the exemplary conditions examined. Other preferred additives include mannitol, sorbitol and N-acetylglucosamine (see, e.g., Examples 5, 6, 13); lactose, dextran-40, PEG 4000, carboxymethylcellulose, TRITON™-x-100, and glycerol (see, e.g., Example 16); trehalose and dextrose (see, e.g., Example 17).

Generally, a huge quantity of the stabilizing additive is not required to confer a notable stabilizing effect. For example, beneficial stabilizing effects were observed at amounts as low as 1% wt/wt stabilizing additive. In general, the amount of stabilizing additive as provided above will range from about 0.25% to about 20%. Exemplary amounts, on a weight percent basis, will range from about 0.5 wt % to about 10 wt %, or more preferably from about 1 wt % to about 5% of a stabilizing additive selected from sucrose, dextran-40, carboxymethylcellulose, mannitol, sorbitol, n-acetylglucosamine, dextrose, trehalose, maltose, lactose, polyethylene glycol,TRITON™-X-100 and glycerol. Illustrative amounts of the foregoing in the instant compositions include 0.5% (all wt %), 1.0%, 1.5%, 2%, 2.5%, 3%, 3.5%, 4%, 4.5%, 5%, 7%, 8%, and 10%. See, e.g., Example 22, in which 1% sucrose was effect to provide a stabilization ratio at 6 months (40° C.) of 7.47.

### Viscosity and Other Measures

Although viscosity can be used an an indication of stability of the hyaluronic-acid polymer based compositions, other rheological measures can be suitably used as well, such as elastic modulus, and change in molecular weight as measured by gel permeation chromatography (GPC) to show stability. Generally, in the instant examples, viscosity was measured using a rheometer under the conditions set forth in the Examples. The viscosity was measured under various conditions of temperature (as an indication of heat stability), humidity, and time, for example, to provide an indication of shelf-life. As can be seen from the examples, the stabilizing additives described herein were effective to notably prevent a significant loss of viscosity over time in the exemplary hyaluronic acid-based corticosteroid compositions examined under a variety of conditions.

### Exemplary Compositions and Methods

Generally speaking, the compositions and methods provided herein are effective to stabilize a hyaluronic acid-based polymer composition comprising a corticosteroid against a reduction in viscosity over time. The stabilization may be measured in any of a number of different ways, for instance, by providing a composition that exhibits a reduction in viscosity of no more than about 50% in comparison to its initial value when stored at 60° C. for a period of 6 days. Alternatively, the composition may exhibit a reduction in viscosity of no more than about 45% in comparison to its initial value when stored at 60° C. for a period of 6 days. Preferably, the composition exhibits a reduction in viscosity of no more than about 40% in comparison to its initial value when stored at 60° C. for a period of 6 days. Even more preferably, the composition exhibits a reduction in viscosity of no more than about 40% in comparison to its initial value when stored at 60° C. for a period of 6 days. Any suitable threshold for assessing an advantageous stabilization may be employed, as can be seen in the accompanying examples. The selection of storage at 60° C. for a period of 6 days (i.e., under accelerated storage conditions) is a convenient measure, as it provides an assessment of formulation stability in a relatively short duration of time. However, as can be seen from the examples, enhanced stability can be measured at any of a number of temperatures, e.g., 60° C., 55° C., 40° C., 45° C., 80° C., and the like, and over any representative period of time, e.g., day 1, day 3, day 5, day 6, day 7, day 10, day 14, day 21, 1 month, 2 months, 3 months, or 6 months, or relative humidites, e.g., typically in a range from about 30% to 75% relative humidity.

In an alternative approach for illustrating the stabilizing advantage of incorporation of a stabilizing additive as described herein, a composition as provided herein will generally possess a stabilization ratio of greater than 1.2. As described previously, the stabilizing ratio is the ratio of the percent retention of viscosity for the hyaluronic acid polymer-corticosteroid composition comprising the stabilizing additive to the percent retention of viscosity of the same hyaluronic acid polymer-corticosteroid composition absent the stabilizing additive, when measured at a given time point and under a defined set of storage conditions. Thus, a composition that possesses a stabilization ratio of 1.0 exhibits a reduction in viscosity that is the same as the composition absent the stabilizing additive, that is to say, the additive fails to provide a measurable degree of protection against a reduction in viscosity over time. Preferably, a composition as provided herein will possess a stabilization ratio of at least 1.3, or of at least 1.5. Particularly preferred compositions possess a stabilization ratio greater than about 1.8. Exemplary stabilization ratios as provided in the accompanying examples include the following values: about 1.5, about 1.6, about 1.7, about 3.0, about 4, about 5.0, about 7, about 7.5, or even greater. Thus, the compositions and methods provided herein are suitable to provide an aqueous hyaluronic acid polymer composition having a stabilization ratio corresponding to any one or more of the values described above or lying within a range between any two of the above stabilization ratio values. The stabilization ratio may be determined at any suitable timepoint, e.g., at day 1, day 3, day 5, day 6, day 7, day 10, day 14, day 21, 1 month, 2 months, 3 months, or 6 months, and at any suitable storage temperature, e.g., 25° C., 40° C., 45° C., 55° C., 60° C., or 80° C., typically at a relative humidity in a range from about 30% to 75% (e.g., at any one of the following: 30% RT, 35% RT, 40% RT, 45% RT, 50% RT, 55% RT, 60% RT, 65% RT, 70% RT, and 75% RT). For example, in one embodiment, the stabilizing ratio is measured at day 6 upon storage of the composition at 60° C.

Also provided is a method of making a hyaluronic acid-based polymer composition having a diminished reduction of viscosity over time, the method comprising incorporating into an aqueous composition comprising a hyaluronic acid polymer and a corticosteroid, a stabilizing amount of a stabilizing additive selected from the group consisting of sugars, sugar alcohols, polyols and polyol-containing surfactants, to thereby provide a composition having a stabilization ratio of greater than 1.2, or alternatively, that exhibits a reduction in viscosity of no more than about 50% in comparison to its initial value when stored at 60° C. for a period of 6 days. In the method, the composition may, for example, be an aqueous solution of the hyaluronic acid polymer, which may be hyaluronic acid per se, or a hyaluronic acid-based polymer, or the composition may comprise an aqueous solution of a hyaluronic acid-based polmer, which may be hyaluronic acid, in combination with a hyaluronic acid polymer-based hydrogel. The aqueous composition may comprise various salts such as sodium chloride, various buffers such as sodium phosphate, and the like. Generally, aqueous compositions comprising the hyaluronic acid polymer, the stabilizing additive, and the corticosteroid are mixed, for example for a period of a few minutes to several hours. Samples of the resulting composition are then generally transferred to containers, such as sterile glass vials, sealed, and placed under storage conditions of choice, as described above and in the examples. Prior to storage, the viscosity of the aqueous solution is measured (e.g., at time equal to zero), to provide an initial value to provide a basis for comparison.

Particularly effective aqueous compositions as provided herein are those comprising sucrose as the stabilizing additive. Illustrative amounts of sucrose in the aqueous compositions are 1% wt/wt, 2% wt/wt, 3% wt/wt, 4% wt/wt, 5% wt/wt, 6% wt/wt, 7% wt/wt, 8% wt/wt, 9% wt/wt, and 10% wt/wt. Indeed, sucrose was found to be particularly effective at stabilization, even at low concentrations such as 1%, 1.25%, and 1.75% (wt/wt). Thus, compositions in accordance with this disclosure may contain any one of the illustrative amounts of the stabilizing additive, sucrose, or an amount falling between any two of the weight percentages provided above.

Additionally, mannitol was found to be particularly effective at preventing a reduction in viscosity, even at concentrations such as 1% wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt. Thus, compositions in accordance with this disclosure may contain any one of the illustrative amounts of the stabilizing additive, mannitol, or an amount of mannitol falling between any two of the weight percentages provided above.

Additional stabilizing additives were also found to be particularly effective at preventing a reduction in viscosity, e.g., sorbitol (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), N-acetylglucosamine (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), lactose (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), dextran-40 (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), PEG 4000 (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), carboxymethylcellulose (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), TRITON-x-100 (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), glycerol (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), trehalose (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt), and dextrose (e.g., at concentrations of % wt/wt, 2% wt/wt, 3% wt/wt, 4% wt/wt, or even greater, e.g., 5% wt/wt, 6% wt/wt, 7% wt/wt, 8% wt/wt, 9% wt/wt, and 10% wt/wt). Thus, additional compositions in accordance with this disclosure may contain any of the illustrative additives described above, in a quantity as described, or in an amount of falling between any two of the weight percentages provided above.

Exemplary compositions are provided below. In one aspect, provided is an aqueous composition comprising a hyaluronic-acid based polymer, a corticosteroid and a stabilizing additive as described above. In one embodiment, the aqueous composition is a solution. Illustrative examples of hyaluronic acid polymer based solutions are provided in Examples 1-22. In yet another embodiment, the aqueous composition comprises, for the hyaluronic acid polymer component, a solution of a hyaluronic acid-based polymer and a hyaluronic acid polymer-based gel, where representative examples of the foregoing are provided in Examples 23-27.

A preferred aqueous solution is a solution comprising unmodified hyaluronic acid, e.g., in saline, where exemplary aqueous solutions of hyaluronic acid have concentrations ranging from about 0.3% to about 4%, or from about 0.5% to about 2% by weight. Alternatively, the aqueous solution may comprise any suitable hyaluronic acid-based polymer as provided herein. The aqueous solution or solution-gel combination further comprises a corticosteroid as described above, and a stabilizing amount of a stabilizing additive (e.g., a sugar, sugar alcohol, polyol or derivatized polyol) as provided herein.

In a particular embodiment, the corticosteroid is selected from from the group consisting of hydrocortisone, hydrocortisone acetate, cortisone acetate, tixocortol pivalate, prednisolone, methylprednisolone, prednisone, triamcinolone, triamcinolone acetonide, triamcinolone benetonide, triamcinolone furetonide, triamcinolone hexacetonide, triamcinolone diacetate, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, beclomethasone dipropionate monohydrate, flunisolide, fluticasone propionate, mometasone furoate monohydrate, and fluticasone furoate.

In one or more preferred embodiments, the corticosteroid is triamcinolone or a derivative of triamcinolone such as triamcinolone acetonide.

In one or more particularly preferred embodiments, the corticosteroid is triamcinolone acetonide.

In yet a further embodiment, the aqueous composition comprises, on a weight percent basis, from about 0.5 wt % to about 10 wt %, or more preferably from about 1 wt % to about 5% of the stabilizing additive.

In yet another embodiment related to any one or more of the foregoing, the stabilizing additive is selected from the group consisting of sucrose, dextran-40, carboxymethylcellulose, mannitol, sorbitol, n-acetylglucosamine, dextrose, trehalose, maltose, lactose, polyethylene glycol,TRITON™-X-100 and glycerol.

In yet one or more preferred embodiments related to the foregoing, the stabilizing additive is sucrose.

Regarding aqueous compositions comprising a hyaluronic acid polymer-based gel, e.g., generally in combination with unmodified hyaluronic acid as described above, illustrative compositions are as provided herein. In a preferred embodiment, the hyaluronic acid gel is hyaluronic acid having 10% or less of its hydroxyl groups derivatized by reaction with divinyl sulfone, which is crosslinked with a thiol crosslinker having two or more thiol groups. In a particularly preferred embodiment, the hyaluronic acid having 10% or less of its hydroxyl groups derivatized by reaction with divinyl sulfone is crosslinked with PEG-dithiol. For example, the degree of conversion of the hydroxyl groups of the hyaluronic acid to 2-(vinylsulfonyl)ethoxy groups is generally selected from 1%, 2%, 3%, 4%, 5%, 6%, 7%, 8%, 9%, and 10%. See, for example, Examples 28 and 29 herein. When in gel form, the hyaluronic acid polymer component in the instant compositions may be in an aqueous composition such as an aqueous slurry, or may be combined with a solution of unmodified hyaluronic acid (see, e.g., Example 31). A preferred aqueous solution is a saline solution of hyaluronic acid, where exemplary aqueous solutions of hyaluronic acid added to the hydrogel have concentrations ranging from about 0.3% to about 4%, or from about 0.5% to about 2% by weight. One representative formulation comprises the following relative amounts of hyaluronic acid polymer-based components: 4 mL of gel slurry ((2-(vinylsulfonyl)ethoxy)_{1-10%}hyaluronic acid/PEG-dithiol) with 2 mL of hyaluronic acid at a concentration of 20 mg/mL. A particularly preferred formulation comprises 4 mL of gel slurry ((2-(vinylsulfonyl)ethoxy)_{4%} hyaluronic acid/PEG-dithiol) with 2 mL of hyaluronic acid at a concentration of 20 mg/mL. Typically, the final hyaluronic acid content in the resulting swollen gel ranges from about 0.05 to 5 percent (0.5 mg/mL to 50 mg/mL). Preferably, the final hyaluronic acid content in the resulting swollen gel is from about 0.1 to 3 percent, or from about 0.1 to 1 percent, or from about 0.5-0.8%. Illustrative final hyaluronic acid content in the resulting swollen gel may, for example, correspond to any of the following percentages: 0.1, 0.2, 0.3, 0.4, 0.5, 0.6, 0.7, 0.8, 0.9, 1.0, 2.0, 3.0, 4.0, and 5.0. For example, representative relative amounts (weight ratios) of hyaluronic acid to crosslinked (e.g., (2-(vinylsulfonyl)ethoxy)_{1-10%} hyaluronic acid/PEG-dithiol) hydrogel particles in the resultant composition typically fall within a range from about 10:1, or from about 5:1, or from about 3:1, or from about 1:1.

Particularly preferred formulations and related methods are those comprising a hyaluronic acid polymer component as set forth above, triamcinolone acetonide and sucrose.

In one or more embodiments of the compositions and methods provided, the compositions are sterile. Additionally, the compositions can be packaged and stored, e.g., in a syringe, for later use.

### Uses

The stabilized hyaluronic acid polymer—corticosteroid compositions described herein may be used in a number of applications, e.g., for wound healing, angiogenesis and tumorigenesis. See D. D. Allison and K. J. Grande-Allen, Tissue Engineering, Vol. 12, Number 8, 2131-2140 (2006); G. D. Prestwich et al, Tissue Engineering, Vol. 12, Number 8, 2171-2180 (2006); G. D. Prestwich et al, Tissue Engineering, Vol. 12, Number 12, 3405-3416 (2006). The stabilized compositions may also be used as tissue fillers, dermal fillers, bulking agents, e.g., as a urethral or a esophageal bulking agent, and embolic agents as well as agents to repair cartilage defects/injuries and agents to enhance bone repair and/or growth. Advantageously, the compositions, based upon the inclusion of a stabilizing additive, will maintain their viscosity to a greater extent than they otherwise would in the absence of such an additive, to provide therapeutic compositions for administration that will ideally persist at a target site for a prolonged extended period of time.

The compositions may also be used in the treatment of osteoarthritis or rheumatoid arthritis, or for other inflammatory arthritis such as gout or calcium pyrophosphate deposition disease (e.g., by injection into the intra-articular space of a joint), or in the reduction or prevention of adhesions that can form following a surgical procedure. The compositions are especially useful for treating chronic or acute inflammation. In particular, the subject compositions are useful for reducing the damage to cartilage upon injection of a corticosteroid by incorporating the corticosteroid into a hyaluronic acid polymer. For instance, in reference to gel-comprising formulations, the incorporation of the corticosteroid is effective to prevent direct contact of the majority of the steroid particles with the joint tissues. Moreover, the trapping of steroid particles in a hyaluronic-acid polmer based hydrogel is effective to maximize the localized concentration of the steroid in the joint, while minimizing its systemic concentration. Additionally, the entrapment of steroid particles in a hydrogel formulation as provided herein is effective to protect the steroid particles from premature clearance from the joint. Finally, by entrapping the steroid in ahydrogel, therapeutic efficacy of the steroid is attained at a lower total dose than would be attained absent hydrogel entrapment, while minimizing unwanted local and systemic side effects.

The compositions may also be used in the treatment of ocular dieseases, in acute gouty arthritis, acute and subacute bursitis, acute nonspecific tenosynovitis, epicondylitis, or synovitis. The compositions can be used in the treatment of alopecia areata; discoid lupus erythematosus; keloids; localized hypertrophic, infiltrated, inflammatory lesions of granuloma annulare, lichen planus, lichen simplex chronicus (neurodermatitis), and psoriatic plaques; necrobiosis lipoidica diabeticorum. It may also be useful in cystic tumors of an aponeurosis or tendon (ganglia).

The present application will now be described in connection with certain embodiments, which are not intended to limit the scope of the invention. On the contrary, the present application covers all alternatives, modifications, and equivalents as included within the scope of the claims. Thus, the following will illustrate the practice of the present application, for the purposes of illustration of certain embodiments and is presented to provide what is believed to be a useful and readily understood description of its procedures and conceptual aspects.

### EXAMPLES

The following examples are put forth to provide those of ordinary skill in the art with a complete disclosure and description of how the compounds, compositions, and methods provided herein are made and evaluated, and are intended to be purely exemplary. Thus, the examples are in no way intended to limit the scope of what the inventors regard as their invention. There are numerous variations and combinations of reaction conditions, e. g., component concentrations, desired solvents, solvent mixtures, temperatures, pressures, and other reaction parameters and conditions that may be employed to optimize product characteristics such as purity, yield, and the like. Such are considered as well within the scope of the present disclosure. Any combination of the above-described elements in all possible variations thereof is encompassed by the invention unless otherwise indicated herein or otherwise clearly contradicted by context.

### Example 1A

### Observed Enhanced Reduction in Viscosity for Crosslinked Hyaluronic Acid-Based Composition Containing a Corticosteroid

During studies on a formulation that contained a crosslinked hyaluronic acid based hydrogel that was suspended in a hyaluronic acid solution, it was observed that in the presence of the corticosteroid, triamcinolone acetonide (TA), the viscosity of the hyaluronic acid component decreased more rapidly over time as compared to the same formulation in the absence of the corticosteroid, triamcinolone acetonide, when stored at 25° C.

TABLE 1

Percent
specific viscosity
retention

| Time | | Formulation 1 + |
|---|---|---|
| (months) | Formulation 1 | TA |
| 0 | 100 | 100 |
| 3 | 97.0 | 78.8 |
| 6 | 94.5 | 68.2 |

Based on the observations summarized in Table 1, additional studies were undertaken to determine whether the loss of viscosity described above could be ameliorated, e.g., by various routes including the incorporation of potentially stabilizing additives.

### Example 1B

### Effect of Sucrose on HA Viscosity at 80° C.

6.9 g of hyaluronic acid, HA (Lifecore, 888 KDa) was dissolved in 881 mL of 0.9% NaCl overnight. The dissolved HA was sterile filtered with 0.2 μm filter (Millipore, Opticap 4″). 3.97 mL of 1 M sodium phosphate, pH 6.71 was added to the HA solution. Then 372 mg of USP grade triamcinolone acetonide (TA) was mixed with 223.2 mL of dissolved HA in a sterile 250 mL Nalgene bottle. Approximately 1.5 g sucrose was added to individual samples of 74±1 mL of the HA/TA suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 80° C. With the exception of the 0 hour samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on 0 hour. The stabilization ratio at 30 hours is the ratio of the percent retention of HA viscosity with the specific additive to the percent retention of HA viscosity of the sample with no additive.

TABLE 2

| | | Percent retention of | | | | |
|---|---|---|---|---|---|---|
| | | HA viscosity | | | | Stabilization |
| Sample | Additive | 0 h | 10 h | 20 h | 30 h | ratio at 30 h |
| HA | — | 100 | 82.6 | 62.6 | 43.7 | N/A |
| Only | | | | | | |
| HA/TA | — | 100 | 29.5 | 9.5 | 6.5 | 1 |
| | Sucrose 2% | 100 | 79.6 | 51.2 | 34.5 | 5.34 |

The results further demonstrate that the presence of the TA results in increased degradation of the HA as shown by the significant decrease in viscosity. The results also show that the addition of sucrose resulted in a notable reduction of the decrease in viscosity as compared to the HA/TA sample with no sucrose.

### Example 2

### Effect of Sucrose on HA Viscosity at 60° C.

6.9 g of HA (Lifecore, 888 KDa) was dissolved in 881 mL of 0.9% NaCl overnight. The dissolved HA was sterile filtered with 0.2 μm filter (Millipore, Opticap 4″). 3.97 mL of 1 M Sodium Phosphate, pH 6.71 was added to the HA solution. Then 372 mg of USP grade triamcinolone acetonide (TA) was mixed with 223.2 mL of dissolved HA in a sterile 250 mL Nalgene bottle. Approximately 1.5 g sucrose was added to individual samples of 74±1 mL of the HA/TA suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 60° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 3

| | | Percent retention of | | | | Stabilization |
|---|---|---|---|---|---|---|
| | | HA viscosity | | | | ratio |
| Sample | Additive | Day 0 | Day 1 | Day 3 | Day 6 | at Day 6 |
| HA | — | 100 | 93.6 | 90.0 | 76.3 | N/A |
| Only | | | | | | |
| HA/TA | — | 100 | 76.3 | 33.0 | 12.5 | 1 |
| | Sucrose | 100 | 93.8 | 85.4 | 64.7 | 5.16 |
| | 2% | | | | | |

The results show that the presence of the TA results in increased degradation of the HA as shown by the significant decrease in viscosity. The results further show that the addition of sucrose resulted in a significant reduction of the decrease in viscosity as compared to the HA/TA sample with no sucrose.

### Example 3

### Effect of Sucrose on HA Viscosity at 55° C.

6.9 g of HA (Lifecore, 888 KDa) was dissolved in 881 mL of 0.9% NaCl overnight. The dissolved HA was sterile filtered with 0.2 um filter (Millipore, Opticap 4″). 3.97 mL of 1 M Sodium Phosphate, pH 6.71 was added to the HA solution. Then 135.3 mg of USP grade Triamcinolone acetonide (TA) was mixed with 81.2 mL of dissolved HA in a sterile 250 mL Nalgene bottle. Approximately 0.5 g sucrose was added to individual samples of 24±2 mL of the HA/TA suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 55° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 4

| | | Percent retention of | | | | Stabilization |
|---|---|---|---|---|---|---|
| | | HA viscosity | | | | ratio |
| Sample | Additive | Day 0 | Day 1 | Day 3 | Day 6 | at Day 6 |
| HA | — | 100 | 97.2 | 93.2 | 85.4 | N/A |
| Only | | | | | | |
| HA/TA | — | 100 | 88.6 | 55.4 | 27.4 | 1 |
| | Sucrose | 100 | 99.3 | 92.4 | 83.0 | 3.03 |
| | 2% | | | | | |

The results show that the presence of the TA results in increased degradation of the HA as shown by the decrease in viscosity. The results further demonstrate that the addition of sucrose resulted in a notable reduction of the decrease in viscosity as compared to the HA/TA sample with no sucrose, with significant stabilization imparted by the presence of sucrose at each time point, but in particular, at further time points (e.g., day 6 shows a greater difference in viscosity between the non-additive containing composition and the additive-containing composition than day 3, and so forth).

### Example 4

### Effect of Sucrose on HA Viscosity at 40° C.

6.9 g of HA (Lifecore, 888 KDa) was dissolved in 881 mL of 0.9% NaCl overnight. The dissolved HA was sterile filtered with 0.2 μm filter (Millipore, Opticap 4″). 3.97 mL of 1 M sodium phosphate, pH 6.71 was added to the HA solution. Then 372 mg of USP grade triamcinolone acetonide (TA) was mixed with 223.2 mL of dissolved HA in a sterile 250 mL Nalgene bottle. Approximately 1.5 g sucrose was added to individual samples of 74±1 mL of the HA/TA suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Week 0 samples, were placed in an oven set at 40° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Week 0. The stabilization ratio at Week 6 is the ratio of the percent retention of HA viscosity with the specific additive to the percent retention of HA viscosity of the sample with no additive.

TABLE 5

| | | Percent retention of | | | | Stabilization |
|---|---|---|---|---|---|---|
| | | HA viscosity | | | | ratio |
| Sample | Additive | Week 0 | Week 2 | Week 4 | Week 6 | at Week 6 |
| HA | — | 100 | 92.3 | 90.9 | 83.4 | N/A |
| Only | | | | | | |
| HA/TA | — | 100 | 52.8 | 26.3 | 17.3 | 1 |
| | Sucrose | 100 | 88.6 | 82.5 | 74.4 | 4.31 |
| | 2% | | | | | |

The results show that the presence of the TA results in increased degradation of the HA as shown by the decrease in viscosity. The results further demonstrate that the addition of sucrose results in a significant reduction of the decrease in viscosity as compared to the HA/TA sample with no sucrose. The effect is further substantiated at 40° C., and becomes increasingly prominent over time.

### Example 5

### Effect of Various Additives on HA Viscosity at 45° C.

7.83 g of HA (Lifecore, 910 KDa) was dissolved in 1000 mL of 0.9% NaCl overnight. The dissolved HA was sterile filtered with 0.2 um filter (Millipore, Opticap 4″). 4.38 mL of 1 M Sodium Phosphate, pH 6.71 was added to the HA solution. Then 500 mg of USP grade Triamcinolone acetonide(TA) was mixed with 300 mL of dissolved HA in a sterile Nalgene bottle. Approximately 0.37 g sucrose, dextrose, maltose, mannitol, sorbitol and N-acetyl glucosamine were added to individual samples of 37±1 mL of the HA/TA suspension respectively to provide samples that contained approximately 1% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Day 0 samples, were placed in an oven set at 45° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Week 12 is the ratio of the percent retention of HA viscosity with the specific additive to the percent retention of HA viscosity of the sample with no additive.

TABLE 6

| | | Percent retention of | | | | |
|---|---|---|---|---|---|---|
| | | HA viscosity | | | | Stabilization |
| Sample | Additive | Day 0 | Week 2 | Week 4 | Week 12 | ratio at Week 12 |
| HA/ | — | 100 | 34.3 | 13.0 | 7.2 | 1 |
| Tri- | Sucrose 1% | 100 | 76.3 | 57.4 | 29.7 | 4.13 |
| amcinolone | Dextrose 1% | 100 | 24.7 | 10.2 | 5.9 | 0.82 |
| acetonide | Maltose 1% | 100 | 10.6 | 6.6 | 4.6 | 0.64 |
| (TA) | Mannitol 1% | 100 | 73.7 | 42.9 | 32.6 | 4.55 |
| | Sorbitol 1% | 100 | 69.5 | 48.3 | 27.9 | 3.88 |
| | N—Ac-GluN | 100 | 46.9 | 25.6 | 13.0 | 1.82 |
| | 1% | | | | | |

The results show that the addition of sucrose, mannitol, sorbitol and N-acetyl glucosamine result in greater (and substantial) retention of the HA viscosity as compared to the samples without the additive. Moreover, the sugars, dextrose and maltose, were ineffective at stabilizing the hyaluronic acid-triamcinolone acetonide composition, as can be seen when compared to the composition absent additive.

### Example 6

### Effect of Additives on HA Viscosity at 80° C.

7.83 g of HA (Lifecore, 910 KDa) was dissolved in 1000 mL of 0.9% NaCl overnight. The dissolved HA was sterile filtered with 0.2 um filter (Millipore, Opticap 4″). 4.38 mL of 1 M Sodium Phosphate, pH 6.71 was added to the HA solution. Approximately 16 g (±1 g) were aliquoted into 6 sterile nalgene bottles. Sucrose was added to 3 bottles such that the final sucrose content in each bottle was approximately 2% (w/w), 5% (w/w) and 10% (w/w) respectively. To the remaining 3 bottles, sorbitol was added such that the final sorbitol content in each bottle was approximately 2% (w/w), 5% (w/w) and 10% (w/w) respectively. To approximately 8 g of each HA/additive solution, triamcinolone acetonide (USP) was added such that final TA content was approximately 1.67 mg per gram HA/additive solution. A control sample was prepared by taking 8.17 ml of the buffered HA solution and adding triamcinolone acetonide (USP) such that final TA content was approximately 1.67 mg per gram HA solution. The samples were stirred for such that the TA was well dispersed in the solution. Approximately 2 ml of each sample was then transferred to a glass scintillation vial and the screw caps were closed. Half the samples were placed in an oven set at 80° C. for 22 hours. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 2 samples) for the 22 h samples to that of the 0 hour samples. The stabilization ratio at 22 h is the ratio of the percent retention of HA viscosity with the specific additive to the percent retention of HA viscosity of the sample with no additive.

TABLE 7

| | |
|---|---|
| Percent | |
| retention | |
| of HA | |
| viscosity | Stabilization |

| Sample | Additive | 0 h | 22 h | ratio at 22 h |
|---|---|---|---|---|
| HA/TA | — | 100 | 5.2 | 1 |
| | Sucrose 2% | 100 | 43.1 | 8.3 |
| | Sucrose 5% | 100 | 58.0 | 11.2 |
| | Sucrose 10% | 100 | 58.7 | 11.3 |
| | Sorbitol 2% | 100 | 33.4 | 6.4 |
| | Sorbitol 5% | 100 | 34.0 | 6.6 |
| | Sorbitol 10% | 100 | 49.4 | 9.5 |

The results in Table 7 show that the addition of both sucrose and sorbitol resulted in greater retention of the HA viscosity as compared to the samples without either additive.

### Example 7

### Effect of Sucrose on HA Viscosity at 80° C.

6.31 g of HA (Lifecore, Average Mw≈880 KDa) was dissolved in 805.5 mL of 0.9% NaCl overnight. The dissolved HA was sterile filtered with 0.2 um filter (Millipore, Opticap 4″). 3.7 mL of 1 M Sodium Phosphate, pH 6.71 was added to the HA solution. Then approximately 1.23 g mg of USP grade Triamcinolone acetonide (TA) was mixed with 740.5 mL of dissolved HA in a sterile Nalgene bottle. Approximately 0.48 g, 0.96 g, 1.4 g and 1.9 g sucrose was added to individual samples of 48±1 mL of the HA/TA suspension respectively to provide samples that contained approximately 1% (w/w), 2% (w/w), 3% (w/w) and 4% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 80° C. With the exception of the 0 hour samples, at each time point (10 hr, 20 hr and 30 hr), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on 0 hour. The stabilization ratio at 30 h is the ratio of the percent retention of HA viscosity with the specific additive to the percent retention of HA viscosity of the sample with no additive.

TABLE 8

| | | | Percent retention of | | | | |
|---|---|---|---|---|---|---|---|
| | | | HA viscosity | | | | Stabilization |
| Temp | Sample | Additive | 0 h | 10 h | 20 h | 30 h | ratio at 30 h |
| 80° C. | HA/TA | — | 100 | 30.3 | 8.6 | 6.5 | 1 |
| | | Sucrose | 100 | 76.3 | 46.5 | 31.8 | 4.9 |
| | | 1% | | | | | |
| | | Sucrose | 100 | 78.9 | 56.1 | 36.9 | 5.7 |
| | | 2% | | | | | |
| | | Sucrose | 100 | 81.5 | 57.5 | 45.0 | 6.9 |
| | | 3% | | | | | |
| | | Sucrose | 100 | 85.2 | 58.9 | 41.9 | 6.4 |
| | | 4% | | | | | |

The results show that the addition of sucrose resulted in greater retention of the HA viscosity as compared to the samples without sucrose.

### Example 8

### Effect of Sucrose on HA Viscosity at 60° C.

6.31 g of HA (Lifecore, Average Mw≠730 KDa) was dissolved in 805.5 mL of 0.9% NaCl overnight. The dissolved HA was sterile filtered with 0.2 um filter (Millipore, Opticap 4″). 3.7 mL of 1 M Sodium Phosphate, pH 6.71 was added to the HA solution. Then approximately 1.23 g mg of USP grade triamcinolone acetonide (TA) was mixed with 740.5 mL of dissolved HA in a sterile Nalgene bottle. Approximately 0.48 g, 0.96 g, 1.4 g and 1.9 g sucrose was added to individual samples of 48±1 mL of the HA/TA suspension respectively to provide samples that contained approximately 1% (w/w), 2% (w/w), 3% (w/w) and 4% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 60° C. With the exception of the Day 0 samples, at each time point (Day 1, Day 4 and Day 6), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 9

| | | | Percent retention of | | | | |
|---|---|---|---|---|---|---|---|
| | | | HA viscosity | | | | Stabilization |
| Temp | Sample | Additive | Day 0 | Day 1 | Day 4 | Day 6 | ratio at Day 6 |
| 60° C. | HA/TA | — | 100 | 86.8 | 44.0 | 31.4 | 1.0 |
| | | Sucrose | 100 | 98.4 | 82.8 | 76.3 | 2.4 |
| | | 1% | | | | | |
| | | Sucrose | 100 | 98.2 | 84.1 | 84.3 | 2.7 |
| | | 2% | | | | | |
| | | Sucrose | 100 | 97.1 | 90.8 | 85.5 | 2.7 |
| | | 3% | | | | | |
| | | Sucrose | 100 | 100.4 | 88.9 | 84.3 | 2.7 |
| | | 4% | | | | | |

The results show that the addition of sucrose resulted in greater retention of the HA viscosity as compared to the samples without sucrose.

### Example 9

### Effect of Sucrose on HA Viscosity at 55° C.

6.31 g of HA (Lifecore, Average Mw≈880 KDa) was dissolved in 805.5 mL of 0.9% NaCl overnight. The dissolved HA was sterile filtered with 0.2 um filter (Millipore, Opticap 4″). 3.7 mL of 1 M Sodium Phosphate, pH 6.71 was added to the HA solution. Then approximately 1.23 g mg of USP grade Triamcinolone acetonide (TA) was mixed with 740.5 mL of dissolved HA in a sterile Nalgene bottle. Approximately 0.48 g, 0.96 g, 1.4 g and 1.9 g sucrose was added to individual samples of 48±1 mL of the HA/TA suspension respectively to provide samples that contained approximately 1% (w/w), 2% (w/w), 3% (w/w) and 4% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 55° C. With the exception of the Day 0 samples, at each time point (Day 1, Day 5 and Day 7), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 7 is the ratio of the percent retention of HA viscosity with the specific additive to the percent retention of HA viscosity of the sample with no additive.

TABLE 10

| | | | Percent retention of | | | | |
|---|---|---|---|---|---|---|---|
| | | | HA viscosity | | | | Stabilization |
| Temp | Sample | Additive | Day 0 | Day 1 | Day 5 | Day 7 | ratio at Day 7 |
| 55° C. | HA/TA | — | 100 | 90.4 | 51.7 | 34.1 | 1.0 |
| | | Sucrose | 100 | 97.7 | 88.9 | 80.5 | 2.4 |
| | | 1% | | | | | |
| | | Sucrose | 100 | 97.9 | 90.8 | 86.6 | 2.5 |
| | | 2% | | | | | |
| | | Sucrose | 100 | 99.1 | 92.2 | 87.0 | 2.6 |
| | | 3% | | | | | |
| | | Sucrose | 100 | 97.2 | 91.3 | 88.4 | 2.6 |
| | | 4% | | | | | |

The results show that the addition of sucrose resulted in greater retention of the HA viscosity as compared to the samples without sucrose.

### Example 10

### Effect of Sucrose on HA (880) Viscosity at 55° C.

4.7 g of HA (Lifecore, 880 KDa) was dissolved in 600 mL of 0.9% NaCl overnight. The dissolved HA was sterile filtered with 0.2 um filter (Millipore, Opticap 4″). 0.84 mL of 1 M Sodium Phosphate, pH 6.71 was added to 167.51 mL of the filtered HA solution. Then 248.4 mg of USP grade Triamcinolone acetonide (TA) was mixed with 144.2 mL of dissolved HA in a sterile 250 mL Nalgene bottle. Approximately 0.24 g, 0.30 g, 0.36 g, 0.42 g and 0.48 g sucrose was added to individual samples of 24±1 mL of the HA/TA suspension respectively to provide samples that contained approximately 1% (w/w), 1.25% (w/w), 1.5% (w/w), 1.75% and 2% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 55° C. With the exception of the 0 hour samples, at each time point (Day 1, Day 3 and Day 7), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 7 is the ratio of the percent retention of HA viscosity with the specific additive to the percent retention of HA viscosity of the sample with no sucrose.

TABLE 11

| | | Percent retention of | | | | |
|---|---|---|---|---|---|---|
| | | HA viscosity | | | | Stabilization |
| Sample | Additive | Day 0 | Day 1 | Day 3 | Day 7 | ratio at Day 7 |
| HA | — | 100.0 | 96.9 | 95.1 | 88.3 | N/A |
| Only | | | | | | |
| HA/triamcinolone | — | 100.0 | 91.3 | 71.9 | 43.6 | 1.0 |
| acetonide | Sucrose 1% | 100.0 | 98.2 | 91.8 | 84.2 | 1.9 |
| | Sucrose 1.25% | 100.0 | 93.9 | 94.4 | 86.0 | 2.0 |
| | Sucrose 1.5% | 100.0 | 96.9 | 95.6 | 87.8 | 2.0 |
| | Sucrose 1.75% | 100.0 | 95.8 | 95.3 | 86.5 | 2.0 |
| | Sucrose 2% | 100.0 | 95.9 | 93.7 | 88.5 | 2.0 |

The data shows that the presence of the TA results in an increased decrease in the viscosity of the HA compared to the HA sample without the TA. The results show that the addition of the sucrose to the HA/TA mixture results in a decreased reduction in viscosity of the HA compared to the HA/TA mixture without the sucrose.

### Example 11

### Effect of MW

### Effect of Sucrose on HA (730) Viscosity at 55° C.

4.7 g of HA (Lifecore, 730 KDa) was dissolved in 600 mL of 0.9% NaCl overnight. The dissolved HA was sterile filtered with 0.2 um filter (Millipore, Opticap 4″). 0.86 mL of 1 M Sodium Phosphate, pH 6.71 was added to 171.8 mL of the HA solution. Then 248.2 mg of USP grade Triamcinolone acetonide (TA) was mixed with 148.9 mL of dissolved HA in a sterile 250 mL Nalgene bottle. Approximately 0.24 g, 0.30 g, 0.36 g, 0.42 g and 0.48 g sucrose was added to individual samples of 24±1 mL of the HA/TA suspension respectively to provide samples that contained approximately 1% (w/w), 1.25% (w/w), 1.5% (w/w), 1.75% and 2% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 55° C. With the exception of the 0 hour samples, at each time point (Day 1, Day 3 and Day 7), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 7 is the ratio of the percent retention of HA viscosity with the specific additive to the percent retention of HA viscosity of the sample with no sucrose.

TABLE 12

| | | Percent retention of | | | | |
|---|---|---|---|---|---|---|
| | | HA viscosity | | | | Stabilization |
| Sample | Additive | Day 0 | Day 1 | Day 3 | Day 7 | ratio at Day 7 |
| HA | — | 100.0 | 96.2 | 94.5 | 94.0 | N/A |
| Only | | | | | | |
| HA/triamcinolone | — | 100.0 | 93.7 | 81.5 | 61.4 | 1 |
| acetonide | Sucrose 1% | 100.0 | 96.7 | 96.5 | 88.9 | 1.4 |
| | Sucrose 1.25% | 100.0 | 99.4 | 98.2 | 93.7 | 1.5 |
| | Sucrose 1.5% | 100.0 | 95.4 | 95.9 | 93.6 | 1.5 |
| | Sucrose 1.75% | 100.0 | 96.8 | 95.8 | 95.1 | 1.5 |
| | Sucrose 2% | 100.0 | 98.4 | 97.8 | 96.2 | 1.6 |

The data shows that the presence of the TA results in an increased decrease in the viscosity of the HA compared to the HA sample without the TA. The results show that the addition of the sucrose to the HA/TA mixture results in a decreased reduction in viscosity of the HA compared to the HA/TA mixture without the sucrose.

### Example 12

### Effect of Sucrose on HA (>1000) Viscosity at 55° C.

15.66 g of HA (Shiseido, >1000 KDa) was dissolved in 2000 mL of 0.9% NaCl overnight. The dissolved HA was sterile filtered with 0.2 um filter (Millipore, Opticap 4″). 9.4 mL of 1 M Sodium Phosphate, pH 6.71 was added to 1873.9 ml of the the HA solution. Then 256.7 mg of USP grade Triamcinolone acetonide (TA) was mixed with 154 mL of dissolved HA in a sterile 250 mL Nalgene bottle. Approximately 0.24 g, 0.30 g, 0.36 g, 0.42 g and 0.48 g sucrose was added to individual samples of 24±1 mL of the HA/TA suspension respectively to provide samples that contained approximately 1% (w/w), 1.25% (w/w), 1.5% (w/w), 1.75% and 2% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 55° C. With the exception of the 0 hour samples, at each time point (Day 1, Day 3 and Day 7), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 7 is the ratio of the percent retention of HA viscosity with the specific additive to the percent retention of HA viscosity of the sample with no sucrose.

TABLE 13

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 7 | Day 7 |
| HA/ | — | 100.0 | 89.5 | 73.3 | 52.1 | 1 |
| triamcinolone | Sucrose 1% | 100.0 | 94.5 | 92.8 | 85.5 | 1.6 |
| acetonide | Sucrose 1.25% | 100.0 | 94.5 | 93.1 | 85.0 | 1.6 |
| | Sucrose 1.5% | 100.0 | 94.9 | 92.6 | 87.6 | 1.7 |
| | Sucrose 1.75% | 100.0 | 96.5 | 91.9 | 88.1 | 1.7 |
| | Sucrose 2% | 100.0 | 95.8 | 91.5 | 87.9 | 1.7 |

The results show that the addition of the sucrose to the HA/TA mixture results in a decreased reduction in viscosity of the HA compared to the HA/TA mixture without the sucrose.

### Example 13

### Effect of Mannitol on HA Viscosity at 80° C.

6.31 g of HA (Lifecore, Average Mw≈880 KDa) was dissolved in 805.5 mL of 0.9% NaCl overnight. The dissolved HA was sterile filtered with 0.2 um filter (Millipore, Opticap 4″). 3.7 mL of 1 M Sodium Phosphate, pH 6.71 was added to the HA solution. Then approximately 1.23 g mg of USP grade Triamcinolone acetonide (TA) was mixed with 740.5 mL of dissolved HA in a sterile Nalgene bottle. Approximately 0.48 g, 0.96 g, 1.4 g and 1.9 g mannitol was added to individual samples of 24±1 mL of the HA/TA suspension respectively to provide samples that contained approximately 1% (w/w), 2% (w/w), 3% (w/w) and 4% (w/w) mannitol respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 80° C. With the exception of the 0 hour samples, at each time point (10 hr, 20 hr and 30 hr), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on 0 hour. The stabilization ratio at 30 h is the ratio of the percent retention of HA viscosity with the specific additive to the percent retention of HA viscosity of the sample with no additive.

TABLE 14

| | | | | | | | Stabili- |
|---|---|---|---|---|---|---|---|
| | | | Percent retention | | | | zation |
| | | | of HA viscosity | | | | ratio at |
| Temp | Sample | Additive | 0 h | 10 h | 20 h | 30 h | 30 h |
| 80° C. | HA/TA | — | 100 | 30.3 | 8.6 | 6.5 | 1 |
| | | Mannitol 1% | 100 | 79.3 | 47.8 | 34.3 | 5.2 |
| | | Mannitol 2% | 100 | 86.6 | 59.7 | 42.3 | 6.5 |
| | | Mannitol 3% | 100 | 84.6 | 62.7 | 42.2 | 6.5 |
| | | Mannitol 4% | 100 | 85.6 | 66.9 | 43.0 | 6.6 |

The results show that the addition of mannitol resulted in greater retention of the HA viscosity as compared to the samples without mannitol.

### Example 14

### Effect of Mannitol on HA Viscosity at 60° C.

6.31 g of HA (Lifecore, Average Mw≈730 KDa) was dissolved in 805.5 mL of 0.9% NaCl overnight. The dissolved HA was sterile filtered with 0.2 um filter (Millipore, Opticap 4″). 3.7 mL of 1 M Sodium Phosphate, pH 6.71 was added to the HA solution. Then approximately 1.23 g mg of USP grade Triamcinolone acetonide (TA) was mixed with 740.5 mL of dissolved HA in a sterile Nalgene bottle. Approximately 0.48 g, 0.96 g, 1.4 g and 1.9 g mannitol was added to individual samples of 24±1 mL of the HA/TA suspension respectively to provide samples that contained approximately 1% (w/w), 2% (w/w), 3% (w/w) and 4% (w/w) mannitol respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 60° C. With the exception of the Day 0 samples, at each time point (Day 1, Day 4 and Day 6), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 15

| | | | Percent retention | | | | Stabili- |
|---|---|---|---|---|---|---|---|
| | | | of HA viscosity | | | | zation |
| | | | Day | Day | Day | Day | ratio at |
| Temp | Sample | Additive | 0 | 1 | 4 | 6 | Day 6 |
| 60° C. | HA/TA | — | 100 | 86.8 | 44.0 | 31.4 | 1.0 |
| | | Mannitol 1% | 100 | 97.1 | 85.1 | 77.8 | 2.5 |
| | | Mannitol 2% | 100 | 99.1 | 86.0 | 80.4 | 2.6 |
| | | Mannitol 3% | 100 | 97.2 | 87.0 | 81.7 | 2.6 |
| | | Mannitol 4% | 100 | 95.3 | 88.8 | 82.1 | 2.6 |

The results show that the addition of mannitol resulted in greater retention of the HA viscosity as compared to the samples without mannitol.

### Example 15

### Effect of Mannitol on HA Viscosity at 55° C.

6.31 g of HA (Lifecore, Average Mw≈880 KDa) was dissolved in 805.5 mL of 0.9% NaCl overnight. The dissolved HA was sterile filtered with 0.2 um filter (Millipore, Opticap 4″). 3.7 mL of 1 M Sodium Phosphate, pH 6.71 was added to the HA solution. Then approximately 1.23 g mg of USP grade Triamcinolone acetonide (TA) was mixed with 740.5 mL of dissolved HA in a sterile Nalgene bottle. Approximately 0.48 g, 0.96 g, 1.4 g and 1.9 g mannitol was added to individual samples of 24±1 mL of the HA/TA suspension respectively to provide samples that contained approximately 1% (w/w), 2% (w/w), 3% (w/w) and 4% (w/w) mannitol respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 55° C. With the exception of the Day 0 samples, at each time point (Day 1, Day 5 and Day 7), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 7 is the ratio of the percent retention of HA viscosity with the specific additive to the percent retention of HA viscosity of the sample with no additive.

TABLE 16

| | | | Percent retention | | | | Stabili- |
|---|---|---|---|---|---|---|---|
| | | | of HA viscosity | | | | zation |
| | | | Day | Day | Day | Day | ratio at |
| Temp | Sample | Additive | 0 | 1 | 5 | 7 | Day 7 |
| 55° C. | HA/TA | — | 100 | 90.4 | 51.7 | 34.1 | 1.0 |
| | | Mannitol 1% | 100 | 98.5 | 89.4 | 79.9 | 2.3 |
| | | Mannitol 2% | 100 | 98.0 | 92.1 | 87.6 | 2.6 |
| | | Mannitol 3% | 100 | 98.3 | 90.2 | 85.5 | 2.5 |
| | | Mannitol 4% | 100 | 98.8 | 93.1 | 86.3 | 2.5 |

The results show that the addition of mannitol resulted in greater retention of the HA viscosity as compared to the samples without mannitol.

### Example 16

### Effect of Additives on HA Viscosity at 60° C.

3.92 g of HA (Shiseido, IV=2 m³/kg) was dissolved in 500 mL of 0.9% NaCl overnight. The dissolved HA was sterile filtered with 0.2 um filter (Millipore, Opticap 4″). 2.16 mL of 1 M Sodium Phosphate, pH 6.71 was added to the HA solution. Then 344.3 mg of USP grade Triamcinolone Acetonide (TA) was mixed with 206.6 mL of dissolved HA in a sterile 250 mL Nalgene bottle. Approximately 0.48 g lactose, melibiose, dextran 40, polyethylene glycol 4000 (PEG 4000), caboxymethyl cellulose sodium salt (CMC), titron-X 100 and glycerol were mixed with each of 24±1 mL of the HA/TA, respectively for 1 hour to provide samples that contained approximately 2% (w/w) additive. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 60° C. With the exception of the Day 0 samples, at each time point (Day 1, Day 3 and Day 7), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 7 is the ratio of the percent retention of HA viscosity with the specific additive to the percent retention of HA viscosity of the sample with no additive.

TABLE 17

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 7 | Day 7 |
| HA Only | — | 100 | 87.4 | 78.4 | 72.6 | N/A |
| HA/ | — | 100 | 75.5 | 49.0 | 26.6 | 1 |
| triamcinolone | Lactose 2% | 100 | 87.5 | 74.4 | 57.6 | 2.17 |
| acetonide | Melibiose 2% | 100 | 81.6 | 52.0 | 23.1 | 0.87 |
| | Dextran 40 2% | 100 | 88.9 | 80.0 | 64.9 | 2.44 |
| | PEG 4000 2% | 100 | 86.3 | 70.0 | 49.0 | 1.84 |
| | CMC 2% | 100 | 88.8 | 68.5 | 46.7 | 1.76 |
| | Tx100 2% | 100 | 83.9 | 59.4 | 32.6 | 1.23 |
| | Glycerol 2% | 100 | 92.1 | 87.7 | 83.2 | 3.13 |

The results show that the presence of the TA results in increased degradation of the HA as shown by the decrease in viscosity. The results show that the addition of lactose, dextran 40, PEG 40000, CMC, triton x100 and glycerol resulted in a reduction of the decrease in viscosity as compared to the HA/TA sample with no additives.

### Example 17

### Effect of Additives on HA Viscosity at 60° C.

3.92 g of HA (Shiseido, IV=2 m³/kg) was dissolved in 500 mL of 0.9% NaCl overnight. The dissolved HA was sterile filtered with 0.2 um filter (Millipore, Opticap 4″). 2.16 mL of 1 M Sodium Phosphate, pH 6.71 was added to the HA solution. Then 344.3 mg of USP grade Triamcinolone Acetonide (TA) was mixed with 206.6 mL of dissolved HA in a sterile 250 mL Nalgene bottle. Approximately 0.48 g trehalose, sucrose, dextrose, maltose, mannitol, sorbitol and N-acetyl glucosamine were mixed with each of 24±1 mL of the HA/TA, respectively for 1 hour to provide samples that contained approximately 2% (w/w) additive. No additives were added to the control sample. For each large sample, 2±0.2 mL aliquots were placed in a sterile 4 mL glass vial which was then sealed with septum and aluminum cap. All these aliquoted samples, except the 0 hour samples, were placed in an oven set at 60° C. With the exception of the Day 0 samples, at each time point (Day 1, Day 3 and Day 6), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 18

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA Only | — | 100 | 87.4 | 78.4 | 72.6 | N/A |
| HA/ | — | 100 | 74.5 | 52.1 | 31.6 | 1 |
| triamcinolone | Trehalose 2% | 100 | 89.7 | 71.4 | 52.0 | 1.65 |
| acetonide | Sucrose 2% | 100 | 91.5 | 86.6 | 78.8 | 2.50 |
| | Dextrose 2% | 100 | 88.5 | 75.1 | 58.6 | 1.86 |
| | Maltose 2% | 100 | 91.1 | 82.9 | 72.4 | 2.29 |
| | Mannitol 2% | 100 | 92.9 | 89.1 | 83.4 | 2.64 |
| | Sorbitol 2% | 100 | 93.0 | 86.8 | 80.2 | 2.54 |
| | N—Ac-GluN 2% | 100 | 92.1 | 78.4 | 58.2 | 1.84 |

The results show that the presence of the TA results in increased degradation of the HA as shown by the decrease in viscosity. The results show that the addition of trehalose, sucrose, dextrose, maltose, mannitol, sorbitol and N-acetyl glucosamine resulted in a reduction of the decrease in viscosity as compared to the HA/TA sample with no additives.

### Example 18

### Effect of Additives on HA Viscosity at 60° C.

19.6 g of HA (Shiseido, IV=2 m³/kg) was dissolved in 2.5 L of 0.9% NaCl overnight. The dissolved HA was sterile filtered with 0.2 um filter (Millipore, Opticap 4″). 1.15 mL of 1 M Sodium Phosphate, pH 6.71 was added to 228.9 mL of the HA solution. Then 334.9 mg of USP grade Triamcinolone hexacetonide(TH) was mixed with 200.95 mL of dissolved HA in a sterile 250 mL Nalgene bottle. Approximately 0.48 g mannitol, sorbitol, maltose, sucrose, glycerol, dextran 40 and lactose was added to individual samples of 24±1 mL of the HA/TH suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Day 0 samples, were placed in an oven set at 60° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 19

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA Only | — | 100 | 88.8 | 69.3 | 52.7 | N/A |
| HA/ | — | 100 | 76.2 | 58.4 | 41.1 | 1.00 |
| triamcinolone | Mannitol | 100 | 95.9 | 84.1 | 76.9 | 1.87 |
| hexacetonide | Sorbitol | 100 | 90.3 | 74.2 | 62.4 | 1.52 |
| | Maltose | 100 | 52.8 | 20.2 | 17.4 | 0.42 |
| | Sucrose | 100 | 93.0 | 82.5 | 67.8 | 1.65 |
| | Glycerol | 100 | 94.7 | 87.2 | 77.9 | 1.89 |
| | Dextran 40 | 100 | 72.4 | 35.6 | 20.9 | 0.51 |
| | Lactose | 100 | 50.7 | 21.5 | 21.9 | 0.53 |

The results show that the presence of triamcinolone hexacetonide results in a decrease in the viscosity of the HA compared to the HA without the triamcinolone hexacetonide. The addition of mannitol, sorbitol, sucrose and glycerol to the HA/TH samples results is a higher retention of the HA viscosity compared the HA/TH only samples.

### Example 19

### Effect of Additives on HA Viscosity at 60° C.

15.66 g of HA (Shiseido, IV=1.9 m³/kg) was dissolved in 2 L of 0.9% NaCl overnight. The dissolved HA was sterile filtered with 0.2 um filter (Millipore, Opticap 4″). 9.4 mL of 1 M Sodium Phosphate, pH 6.71 was added to 1.87 L of the HA solution. Then 333.3 mg of methyl prednisolone acetate (MPA) was mixed with 200 mL of dissolved HA in a sterile 250 mL Nalgene bottle. Approximately 0.48 g mannitol, sorbitol, maltose, sucrose, glycerol, dextran 40 and lactose was added to individual samples of 24±1 mL of the HA/MPA suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Day 0 samples, were placed in an oven set at 60° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 20

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA/Methyl | — | 100 | 89.9 | 75.5 | 44.6 | 1.00 |
| prednisolone | Mannitol | 100 | 94.5 | 91.0 | 85.8 | 1.92 |
| acetate | Sorbitol | 100 | 95.8 | 90.7 | 82.3 | 1.84 |
| | Maltose | 100 | 93.3 | 84.5 | 69.6 | 1.56 |
| | Sucrose | 100 | 95.1 | 92.1 | 82.5 | 1.85 |
| | Glycerol | 100 | 98.6 | 91.4 | 85.5 | 1.92 |
| | Dextran 40 | 100 | 93.4 | 85.4 | 74.0 | 1.66 |
| | Lactose | 100 | 92.7 | 80.1 | 65.8 | 1.47 |

The addition of mannitol, sorbitol, maltose, sucrose, glycerol, dextran 40 and lactose to the HA/MPA samples results is a higher retention of the HA viscosity compared the HA/MPA only samples. The stabilization ratio at Day 6 is greater than 1 for all the samples with the additives.

### Example 20

### Effect of Additives on HA Viscosity at 60° C.

15.66 g of HA (Shiseido, IV=1.9 m³/kg) was dissolved in 2 L of 0.9% NaCl overnight. The dissolved HA was sterile filtered with 0.2 um filter (Millipore, Opticap 4″). 9.4 mL of 1 M Sodium Phosphate, pH 6.71 was added to 1.87 L of the HA solution. Then 338.9 mg of betamethasone sodium phosphate (BMSP) was mixed with 203.4 mL of dissolved HA in a sterile 250 mL Nalgene bottle. Approximately 0.48 g mannitol, sorbitol, maltose, sucrose, glycerol, dextran 40 and lactose was added to individual samples of 24±1 mL of the HN BMSP suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Day 0 samples, were placed in an oven set at 60° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 21

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA/ | — | 100 | 79.3 | 60.2 | 37.6 | 1.00 |
| betamethasone | Mannitol | 100 | 94.9 | 89.3 | 81.2 | 2.16 |
| sodium | Sorbitol | 100 | 94.2 | 89.3 | 79.2 | 2.11 |
| phosphate | Maltose | 100 | 93.3 | 81.6 | 64.0 | 1.70 |
| | Sucrose | 100 | 93.3 | 88.9 | 78.5 | 2.09 |
| | Glycerol | 100 | 94.5 | 92.5 | 82.0 | 2.18 |
| | Dextran 40 | 100 | 91.9 | 79.0 | 65.3 | 1.74 |
| | Lactose | 100 | 87.5 | 73.0 | 54.0 | 1.44 |

The addition of mannitol, sorbitol, maltose, sucrose, glycerol dextran 40 and lactose to the HA/BMSP samples results is a higher retention of the HA viscosity compared the HN BMSP only samples. The stabilization ratio at Day 6 is greater than 1 for all the samples with the additives.

### Example 21

### Effect of Additives on HA Viscosity at 60° C.

15.66 g of HA (Shiseido, IV=1.9 m³/kg) was dissolved in 2 L of 0.9% NaCl overnight. The dissolved HA was sterile filtered with 0.2 um filter (Millipore, Opticap 4″). 9.4 mL of 1 M Sodium Phosphate, pH 6.71 was added to 1.87 L of the HA solution. Then 356.7 mg of prednisolone acetate (PA) was mixed with 210.4 mL of dissolved HA in a sterile 250 mL Nalgene bottle. Approximately 0.48 g mannitol, sorbitol, maltose, sucrose, glycerol, dextran 40 and lactose was added to individual samples of 24±1 mL of the HA/PA suspension respectively to provide samples that contained approximately 2% (w/w) additive. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Day 0 samples, were placed in an oven set at 60° C. With the exception of the Day 0 samples, at each time point, 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 22

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA/ | — | 100 | 91.6 | 75.8 | 51.0 | 1.00 |
| prednisolone | Mannitol | 100 | 98.6 | 94.2 | 89.1 | 1.75 |
| acetate | Sorbitol | 100 | 97.4 | 91.4 | 87.7 | 1.72 |
| | Maltose | 100 | 96.0 | 86.1 | 73.4 | 1.44 |
| | Sucrose | 100 | 97.5 | 91.7 | 86.7 | 1.70 |
| | Glycerol | 100 | 96.5 | 93.6 | 88.5 | 1.74 |
| | Dextran 40 | 100 | 92.2 | 84.6 | 73.6 | 1.44 |
| | Lactose | 100 | 90.4 | 78.3 | 64.6 | 1.27 |

The addition of mannitol, sorbitol, maltose, sucrose, glycerol dextran 40 and lactose to the HA/PA samples results is a higher retention of the HA viscosity compared the HA/PA only samples. The stabilization ratio at Day 6 is greater than 1 for all the samples with the additives.

### Example 22

### Effect of Surose on HA Viscosity at 40° C.

3.5 g of HA (Lifecore, 730 KDa) was dissolved in 450 mL of 0.9% NaCl overnight. The dissolved HA was sterile filtered with 0.2 um filter (Millipore, Opticap 4″). 1.77 mL of 1 M Sodium Phosphate, pH 6.71 was added to 354 mL of the HA solution. Then 590 mg of USP grade Triamcinolone acetonide (TA) was mixed with 354 mL of dissolved HA in a sterile Nalgene bottle. Approximately 0.587 g sucrose was added to individual samples of 58.7±1 mL of the HA/TA suspension respectively to provide samples that contained approximately 1% (w/w) sucrose respectively. Each mixture was mixed for 1 hour. No additives were added to the control sample. For each large sample, 2±0.2 mL aliquots were placed in a sterile 4 mL glass vial which was then sealed with septum and aluminum cap. All these aliquoted samples, except the Day 0 samples, were placed in an oven set at 40° C. With the exception of the Day 0 samples, at each time point (15 days, 30 days, 45 days and 6 months), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at 6 months is the ratio of the percent retention of HA viscosity with the specific additive to the percent retention of HA viscosity of the sample with no additive.

TABLE 23

| | | Percent retention | | | | | Stabili- |
|---|---|---|---|---|---|---|---|
| | | of HA viscosity | | | | | zation |
| | | Day | 15 | 30 | 45 | 6 | ratio at |
| Sample | Additive | 0 | Days | Days | Days | Months | 6 Months |
| HA/TA | — | 100 | 62.6 | 45.3 | 26.9 | 4.7 | 1 |
| | Sucrose 1% | 100 | 91.0 | 85.3 | 76.6 | 34.8 | 7.47 |

### Example 23

### Effect of Sucrose on HA Viscosity for Hydrogel/HA Solution

Formulation B: 29.19 g vinyl sulfone modified hyaluronic acid (HAVS), (Mw730 KDa, average substitution=3.9%) was dissolved in 1837.6 mL water for injection (WFI). 807.6 g of this HAVS solution was aseptically transferred into a sterile 8 L reaction tanks following double sterile filtration through a 0.2 um filter capsule. 22.7 g of a sterile filtered phosphate (sodium phosphate monobasic monohydrate [USP], 12.4 g in 161 g WFI, pH adjusted to pH7.4 using 6N NaOH and 6N HCL). 9.19 g sterile triamcinolone acetonide (USP) was then added to the solution. The mixture was stirred for 10 minutes at 50 rpm and 2 hours at 150 rpm. 28.4 g sterile filtered dithiol polyethylene glycol 3350 solution (50 mg/mL in WFI) was added to the mixture. The mixture was stirred at 150 rpm for 3 minutes after which water from a circulating water bath (45° C.) was circulated in the reaction tank water jacket. The mixture was then stirred at 50 rpm for 15 min afterwhich the stirrer was turned off. After approximately 17 hours, the water circulation was stopped and the formed gel was broken into smaller pieces by turning on the stirrer for 5 min at 50 rpm. 4,6521 g of a sterile filtered HA/0.9% NaCl solution (7.8 mg/mL, Mw≈730 KDa) was added to the broken gel. After 2 hours, the resultant mixture was aseptically pumped through 2×840 um mesh and into a filling tank. The meshed mixture was stirred at 20 rpm for 3 hours after which the mixture was filled into sterile 10 mL glass syringes using a Baxa repeater pump (approximately 6.2 mL per syringe). The syringes were then stoppered. A portion of the syringes were stored at 40° C./75% RH for 3 month and another portion of the syringes were stored at 25° C./60% RH for 3 months.

Formulation B+sucrose: A formulation was prepared using a similar method described with the exception that the HA/0.9% NaCl solution used contained 1.7% or 2% (w/w) sucrose (USP).

The specific viscosity of the supernatant solution of the samples prepared above was measured using a Cannon-Ubbelohde semi-micro glass viscometer in a water bath set at 25° C. The samples for viscosity measurement were prepared by taking 1 mL of the sample supernatant and diluting it with 7 mL 0.1 M phosphate buffer (sodium phosphate monobasic monohydrate, pH 7). The phosphate buffer was used to measure the solvent flow time. The percent retention of the HA viscosity was determined by the ratio of the specific viscosity (average of the 3 samples) at 3 months to that on Month 0.

TABLE 24
Storage at 40° C./75% RH

| | Percent specific viscosity retention | | |
|---|---|---|---|
| | | Formulation B + | |
| Time | | 1.7% | Formulation B + |
| (Months) | Formulation B | sucrose | 2% sucrose |
| 0 | 100 | 100 | 100 |
| 3 | 40.2 | 66.8 | 81.6 |
| 6 | 31.9 | 55.6 | 68.4 |

TABLE 25
Storage at 25° C./60% RH

| | Percent specific viscosity retention | | |
|---|---|---|---|
| | | Formulation B + | |
| Time | | 1.7% | Formulation B + |
| (Months) | Formulation B | sucrose | 2% sucrose |
| 0 | 100 | 100 | 100 |
| 3 | 80.7 | 96.8 | 96.3 |
| 6 | 68.5 | 94.2 | 89.5 |

The data shows that the addition of sucrose to the formulation results in greater retention of viscosity of the HA component at both 25° C. and 40° C. as compared to the formulation without the added sucrose.

### Example 24

### Effect of Sucrose on HA Viscosity for Hydrogel/HA Solution at 80° C.

4.92 g HAVS (Mw 730-950 KDa; average substitution=3.9%) was dissolved in 351 mL WFI. The solution was then sterile filtered using a 0.2 um filter. Approximately 13.5±0.5 mL of the HAVS solution was aliquoted into 100 mL plastic bottles. A total of 4 bottles were filled. Triamcinolone acetonide (USP) [TA] was added to each aliquot such that the TA content was approximately 1.67 mg/mL. The contents were mixed well. Approximately 0.38 mL 1M phosphate buffer (pH 7.4) was added to each mixture bottle and the content were mixed well. Approximately 0.47 mL of a 50 mg/ml dithiol polyethylene glycol 3350 solution was added to each bottle. The contents were well mixed. The screw caps to the bottles were closed and each solution was incubated overnight at 45° C. A 7.83 mg/mL HA solution (0.9% saline) was prepared and filtered through a 0.2 um filter. 3.4 mL 1M phosphate buffer (pH 6.7) was added to 678 mL of the HA solution. The bottles were removed from the oven and approximately 77±2 mL HA solution (MW=730 KDa; 7.83 mg/mL in 0.9% saline) was added to each bottle. To one of the bottles, sucrose was added to yield a formulation with approximately 2% (w/w) sucrose. To a second bottle, sucrose was added to yield a formulation with approximately 3% (w/w) sucrose. After 1 hour mixing, the contents of each bottle were extruded through a 840 um mesh using a 60 mL syringe and a syringe filter that contained the mesh. Each extruded mixture was aliquoted (Approximately 6.2 mL) into 20 ml glass scintillation vials. The vials were closed with screw cap lids and except for the 0 hour samples, the samples were placed in an oven set at 80° C. With the exception of the 0 hour samples, at each time point (10 hr, 20 hr and 30 hr), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on 0 hour. The stabilization ratio at 30 h is the ratio of the percent retention of HA viscosity with the specific additive to the percent retention of HA viscosity of the sample with no additive.

TABLE 26

| | | | | | | Stabili- |
|---|---|---|---|---|---|---|
| | | Percent retention | | | | zation |
| | | of HA viscosity | | | | ratio at |
| Sample | Additive | 0 h | 10 h | 20 h | 30 h | 30 h |
| HA/TA | — | 100 | 31.7 | 28.7 | 15.9 | 1 |
| | Sucrose 2% | 100 | 72.0 | 51.8 | 30.3 | 1.9 |
| | Sucrose 3% | 100 | 75.3 | 53.0 | 54.3 | 3.4 |

The results show that the addition of sucrose resulted in greater retention of the HA viscosity as compared to the samples without sucrose.

### Example 25

### Effect of Sucrose on HA Viscosity for Hydrogel/HA Solution 80° C.

4.92 g HAVS (Mw 730-950 KDa; average substitution=3.9%) was dissolved in 351 mL WFI. The solution was then sterile filtered using a 0.2 um filter. Approximately 14.2±0.5 mL of the HAVS solution was aliquoted into 100 mL plastic bottles. A total of 4 bottles were filled. Triamcinolone acetonide (USP) [TA] was added to each aliquot such that the TA content was approximately 1.67 mg/mL. The contents were mixed well. Approximately 0.4 mL 1M phosphate buffer (pH 7.4) was added to each mixture bottle and the content were mixed well. Approximately 0.5 mL of a 50 mg/ml dithiol polyethylene glycol 3350 solution was added to each bottle. The contents were well mixed. The screw caps to the bottles were closed and each solution was incubated overnight at 45° C. A 7.83 mg/mL HA solution (0.9% saline) was prepared and filtered through a 0.2 um filter. 3.97 mL 1M phosphate buffer (pH 6.7) was added to 794.7 mL of the HA solution. The bottles were removed from the oven and approximately 82±2 mL HA solution (MW=880 KDa; 7.83 mg/mL in 0.9% saline) was added to each bottle. To one of the bottles, sucrose was added to yield a formulation with approximately 2% (w/w) sucrose. To a second bottle, sucrose was added to yield a formulation with approximately 3% (w/w) sucrose. After 1 hour mixing, the contents of each bottle were extruded through a 840 um mesh using a 60 mL syringe and a syringe filter that contained the mesh. Each extruded mixture was aliquoted (Approximately 6.2 mL) into 20 ml glass scintillation vials. The vials were closed with screw cap lids and except for the 0 hour samples, the samples were placed in an oven set at 80° C. With the exception of the 0 hour samples, at each time point (10 hr, 20 hr and 30 hr), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on 0 hour. The stabilization ratio at 30 h is the ratio of the percent retention of HA viscosity with the specific additive to the percent retention of HA viscosity of the sample with no additive.

TABLE 27

| | | | | | | Stabili- |
|---|---|---|---|---|---|---|
| | | Percent retention | | | | zation |
| | | of HA viscosity | | | | ratio at |
| Sample | Additive | 0 h | 10 h | 20 h | 30 h | 30 h |
| HA/TA | — | 100 | 47.7 | 8.4 | 6.4 | 1 |
| | Sucrose 2% | 100 | 67.3 | 38.4 | 20.5 | 3.2 |
| | Sucrose 3% | 100 | 78.9 | 53.5 | 16.7 | 2.6 |

The results show that the addition of sucrose resulted in greater retention of the HA viscosity as compared to the samples without sucrose.

### Example 26

### Effect of Sucrose on HA Viscosity for Hydrogel/HA Solution 60° C.

4.92 g HAVS (Mw 730-950 KDa; average substitution=3.9%) was dissolved in 351 mL WFI. The solution was then sterile filtered using a 0.2 um filter. Approximately 13.5±0.5 mL of the HAVS solution was aliquoted into 100 mL plastic bottles. A total of 4 bottles were filled. Triamcinolone acetonide (USP) [TA] was added to each aliquot such that the TA content was approximately 1.67 mg/mL. The contents were mixed well. Approximately 0.38 mL 1M phosphate buffer (pH 7.4) was added to each mixture bottle and the content were mixed well. Approximately 0.47 mL of a 50 mg/ml dithiol polyethylene glycol 3350 solution was added to each bottle. The contents were well mixed. The screw caps to the bottles were closed and each solution was incubated overnight at 45° C. A 7.83 mg/mL HA solution (0.9% saline) was prepared and filtered through a 0.2 um filter. 3.4 mL 1M phosphate buffer (pH 6.7) was added to 678 mL of the HA solution. The bottles were removed from the oven and approximately 77±2 mL HA solution (MW=730 KDa; 7.83 mg/mL in 0.9% saline) was added to each bottle. To one of the bottles, sucrose was added to yield a formulation with approximately 2% (w/w) sucrose. To a second bottle, sucrose was added to yield a formulation with approximately 3% (w/w) sucrose. After 1 hour mixing, the contents of each bottle were extruded through a 840 um mesh using a 60 mL syringe and a syringe filter that contained the mesh. Each extruded mixture was aliquoted (Approximately 6.2 mL) into 20 ml glass scintillation vials. The vials were closed with screw cap lids and except for the Day 0 samples, the samples were placed in an oven set at 60° C. With the exception of the Day 0 samples, at each time point (Day 1, Day 3, Day 6), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 28

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA/TA | — | 100 | 64.5 | 55.3 | 26.2 | 1 |
| | Sucrose 2% | 100 | 94.0 | 85.7 | 73.5 | 2.8 |
| | Sucrose 3% | 100 | 94.5 | 81.6 | 76.5 | 2.9 |

The results show that the addition of sucrose resulted in greater retention of the HA viscosity as compared to the samples without sucrose.

### Example 27

### Effect of Sucrose on HA Viscosity for Hydrogel/HA Solution 55° C.

4.92 g HAVS (Mw 730-950 KDa; average substitution=3.9%) was dissolved in 351 mL WFI. The solution was then sterile filtered using a 0.2 um filter. Approximately 14.2±0.5 mL of the HAVS solution was aliquoted into 100 mL plastic bottles. A total of 4 bottles were filled. Triamcinolone acetonide (USP) [TA] was added to each aliquot such that the TA content was approximately 1.67 mg/mL. The contents were mixed well. Approximately 0.4 mL 1M phosphate buffer (pH 7.4) was added to each mixture bottle and the content were mixed well. Approximately 0.5 mL of a 50 mg/ml dithiol polyethylene glycol 3350 solution was added to each bottle. The contents were well mixed. The screw caps to the bottles were closed and each solution was incubated overnight at 45° C. A 7.83 mg/mL HA solution (0.9% saline) was prepared and filtered through a 0.2 um filter. 3.97 mL 1M phosphate buffer (pH 6.7) was added to 794.7 mL of the HA solution. The bottles were removed from the oven and approximately 82±2 mL HA solution (MW=880 KDa; 7.83 mg/mL in 0.9% saline) was added to each bottle. To one of the bottles, sucrose was added to yield a formulation with approximately 2% (w/w) sucrose. To a second bottle, sucrose was added to yield a formulation with approximately 3% (w/w) sucrose. After 1 hour mixing, the contents of each bottle were extruded through a 840 um mesh using a 60 mL syringe and a syringe filter that contained the mesh. Each extruded mixture was aliquoted (Approximately 6.2 mL) into 20 ml glass scintillation vials. The vials were closed with screw cap lids and except for the Day 0 samples, the samples were placed in an oven set at 55° C. With the exception of the Day 0 samples, at each time point (Day 1, Day 3, Day 6), 3 of the samples were removed from the oven and allowed to cool to room temperature. The viscosity of each sample was measured using AR550 Rheometer (TA Instrument) with 40 mm 2° steel cone at 22° C. with a 3 min time sweep. The percent retention of the HA viscosity was determined by the ratio of the viscosity (average of the 3 samples) on the specified day to that on Day 0. The stabilization ratio at Day 6 is the ratio of the percent retention of HA viscosity with the specific additive to the percent retention of HA viscosity of the sample with no additive.

TABLE 29

| | | Percent retention | | | | Stabili- |
|---|---|---|---|---|---|---|
| | | of HA viscosity | | | | zation |
| | | Day | Day | Day | Day | ratio at |
| Sample | Additive | 0 | 1 | 3 | 6 | Day 6 |
| HA/TA | — | 100 | 72.8 | 42.7 | 21.0 | 1 |
| | Sucrose 2% | 100 | 88.6 | 87.7 | 76.3 | 3.6 |
| | Sucrose 3% | 100 | 86.7 | 84.9 | 80.0 | 3.8 |

The results show that the addition of sucrose resulted in greater retention of the HA viscosity as compared to the samples without sucrose.

### Example 28

### Synthesis of Vinyl Sulfone Derivatized Hyaluronic acid (HA-VS)—Low Degree of Modification

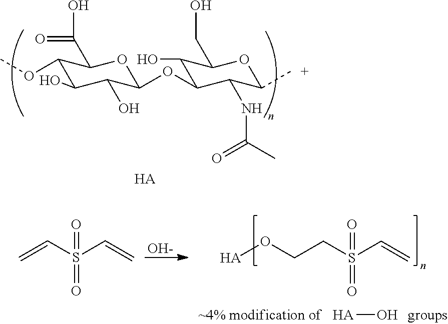

This example describes the preparation of an exemplary and preferred material for the stabilized compositions provided herein.

5 g hyaluronic acid (HA) [9.4×10⁴ cps (3% in water)] was weighed into a 1 L round bottom flask. 500 mL sterile filtered water was added to the HA. The flask was placed on a rotary evaporator which was set to rotate at between 20-100 rpm. The solution was rotated until all the HA was dissolved (approx. 16-18 hrs). The HA solution (10 mg/mL) was then transferred to a 1 L glass beaker. A stirring paddle that was connected to an overhead stirrer was inserted into the solution and was set to a stirring speed that ensured efficient stirring of the solution. 333 mL of a 0.25 N NaOH solution (83.2 mL 1N NaOH added to 249.8 mL deionized water) was added to the stirring HA solution. After about 1 min, 150 mL of a divinyl sulfone solution (18 mL divinyl sulfone dissolved in 132 mL deionized water) was added rapidly to the stirring solution. After 15 seconds (as measured from the completion of the divinyl sulfone solution addition), the pH of the solution was adjusted to between 5 and 6 by rapidly adding approx. 14 mL 6N HCl. The reaction solution was then dialysed using a tangential flow filtration system (spectrapor system, cartridge P/N M6-100S-301-01P). The total volume was 11 times the original solution volume. Once the purification step was completed, the solution was concentrated to approx. 14-20 mg/mL. The vinyl sulfone functionalized HA (HA-VS) was removed from the TFF system and was placed in a plastic container which was then closed with a screw top lid. A sample of the HA-VS was removed, frozen at −80° C. and then lyophilized. The dried sample was sent for ¹H-NMR analysis.

Determination of Percentage Vinyl Sulfone Substitution for HA-VS

Approx. 15-17 mg of the dried sample was weighed into a tared 2 mL tube. The sample was reconstituted in 1.5 mL D₂O. The sample was transferred to an NMR tube. The ¹H-NMR (256 scans) of the sample was taken and the spectrum was printed out with the specific peaks in the 6.3-6.5 ppm (2 peaks from the 2 CH₂=protons from the vinyl sulfone residue) and 1.5-2.5 ppm (singlet from the 3 CH₃-protons from the N-acetyl group of the HA) regions being integrated. The percent modification is calculated as follows:

$$\% modification = 150 \times \frac{Integral vinyl sulfone peaks}{Intergral Acetamide peak}$$

The ¹H-NMR spectrum (FIG. 1) showed that the HA had a vinyl sulfone substitution level of approximately 4%, based upon an integration of vinyl sulfone peaks relative to the acetamide methyl group of the hyaluronic acid.

A sample of the HA-VS was used to determine the dry weight which was used to determine the specific concentration of the HA-VS solution. The HA-VS concentration was 18 mg/mL.

### Example 29

### Synthesis of a Gel Prepared from Vinyl Sulfone Modified Hyaluronic Acid (HA-VS) and PEG-3400-Dithiol

A solution of HA-VS, prepared as described in Example 1, was diluted using deionized water to a concentration of 14 mg/mL. 11 mL of the HA-VS solution was placed into a 20 mL sterile syringe. The HA-VS solution was filtered through a 0.2 μm sterile syringe filter into a sterile 50 mL centrifuge tube. A 50 mg/mL solution of PEG-(SH)₂ [Laysan Bio Inc, Item# SH-PEG-SH-3400-1 g] was prepared by dissolving 40.1 mg PEG-(SH)₂ in 0.802 mL deionized water. The PEG-(SH)₂ solution was transferred to a 1 mL sterile syringe and was filtered through a 0.2 μm sterile syringe filter. 10 mL of the sterile filtered HA-VS was transferred to a sterile 50 mL centrifuge tube. 250 μL of a 0.5 M sodium phosphate solution (filtered through a 0.2 μm sterile syringe filter) was added to the HA-VS solution. The resultant solution was mixed thoroughly. 380 μL [19 mg PEG-(SH)₂] of the sterile 50 mg/mL PEG-(SH)₂ solution was added to the HA-VS solution. The resultant solution was mixed thoroughly. The above steps were performed in a biohood to maintain sterility. The HA-VS/PEG-(SH)₂ solution was then placed in a 37° C. incubator for at least 16 hours to promote gel formation. After at least 16 hours, the HA-VS/PEG-(SH)₂ solution had crosslinked to form a gel. The gelled material was then removed from the incubator.

### Example 30

### Preparation of a HA-VS/PEG-(SH)₂ Gel Slurry—Single Extrusion

The HA-VS/PEG-(SH)₂ gel from Example 29 was physically broken into pieces using a glass rod. The gel was transferred to a sterile 60 mL syringe that was capped with a syringe cap. 40 mL 0.9% sterile NaCl was added to the gel. The plunger was inserted into the syringe barrel and the syringe was inverted. The syringe cap was opened to release any pressure and was then closed. The syringe was inverted several times to ensure good mixing of the saline and the gel pieces. The gel was allowed to swell overnight (at least 16 hrs).

A 23 mm diameter disc of a polyester mesh (McMaster Carr, Cat #9218T13, Mesh Size: 20.3×20.3, Square/Rectangle Size: 0.0331″, Micron Rating: 840 Microns, Percentage of Open Area: 46, Thread Diameter: 0.0157″) was cut out using a 23 mm leather punch. The disc was inserted into a 25 mm syringe filter holder (Cole Palmer, Cat #EW-29550-42) and the filter holder was closed. The filter holder that contained the mesh was autoclaved. The syringe cap of the syringe was removed and the syringe filter containing the mesh was attached to the syringe. The gel was extruded through the mesh into a sterile 50 mL centrifuge tube. The centrifuge tube was capped with a screw top lid. The resulting product is a slightly viscous slurry of the particles, where the particles do not really settle out but typically remain suspended. The above steps were performed in a biohood.

### Example 31

### Preparation of HA-VS/PEG-(SH)₂ Gel Slurry with Hyaluronic Acid

2 g hyaluronic acid [9.4×10⁴ cps (3% in water)] was weighed into a 250 mL round bottom flask. 100 mL sterile saline was added to the hyaluronic acid in the flask. The flask was attached to a rotary evaporator (Buchi) and was rotated at 50 rpm for at least 16 hrs to form a 2% hyaluronic acid solution. The following series of steps were performed in a biohood. The hyaluronic acid solution was filtered through a 0.2 um sterile filter. Using the HAVS/PEG-(SH)2 gel slurry, a series of formulations were prepared in which the prepared HA-VS/PEG-(SH)2 gel slurry was mixed with hyaluronic acid. The volumes of the hyaluronic acid solution and the HA-VS/PEG(SH)₂ gel slurry used to prepare these formulations are shown in the table below:

TABLE 30

| | VOLUME | |
|---|---|---|
| | HYALURONIC | VOLUME HA-VS/PEG- |
| FORMULATION | ACID (ML) | (SH)₂ GEL SLURRY (ML) |
| 1 | 1 | 5 (single extrusion slurry) |
| 2 | 2 | 4 (single extrusion slurry) |
| 3 | 3 | 3 (single extrusion slurry) |
| 4 | 4 | 2 (single extrusion slurry) |
| 5 | 5 | 1 (single extrusion slurry) |
| 6 | 1 | 5 (double extrusion slurry) |
| 7 | 2 | 4 (double extrusion slurry) |
| 8 | 3 | 3 (double extrusion slurry) |
| 9 | 4 | 2 (double extrusion slurry) |
| 10 | 5 | 1 (double extrusion slurry) |

The indicated volumes of hyaluronic acid solution and HA-VS/PEG-(SH)2 gel slurry, as identified in the table above, were added to a 15 mL sterile centrifuge tube. The cap was placed on the tube and the tube was inverted back and forth until the components were well mixed. Each formulation was then transferred into a 10 mL glass syringe that had a syringe cap after which the plunger was inserted and the excess air was expelled. The syringe cap was then tightened.

### Example 32

### Synthesis of HA-VS-PEG-(SH)₂ Gel Containing Triamcinolone Acetonide

A solution of the HA-VS, was diluted using deionized water to a concentration of 14 mg/mL. 11 mL HA-VS solution was placed into a 20 mL sterile syringe. The HA-VS solution was filtered through a 0.2 um sterile syringe filter into a sterile 50 mL centrifuge tube. A 50 mg/mL solution of PEG-(SH)₂ was prepared by dissolving 40.1 mg PEG3400-(SH)₂ in 0.802 mL deionized water. The PEG-(SH)₂ solution was transferred to a 1 mL sterile syringe and was filtered through a 0.2 um sterile syringe filter. 10 mL of the sterile filtered HA-VS was transferred to a sterile 50 mL centrifuge tube. 100 mg of triamcinolone acetonide (Spectrum Chemicals, U.S.P grade, micronized) was added to the HAVS solution. The cap of the centrifuge tube was placed on the tube and the solution was inverted back and forth until the triamcinolone acetonide was homogeneously mixed with the HA-VS. 250 μL of a sterile filtered (0.2 um sterile filter) 0.5 M sodium phosphate solution was added to the HA-VS solution. The resultant solution was mixed thoroughly. 380 μL of the sterile 50 mg/mL PEG-(SH)₂ solution was added to the HA-VS solution. The resultant solution was mixed thoroughly. The above steps were performed in a biohood. The HA-VS/PEG-(SH)₂ solution was then placed in a 37° C. incubator for at least 16 hours. At this stage the HA-VS/PEG(SH)₂ solution had crosslinked to form a gel. The gelled material was then removed from the incubator. The resulting gel contains approximately 0.2% triamcinolone acetonide.

The above procedure was also carried out as set forth above with the exception that 20 mg of triamcinolone acetonide (Spectrum Chemicals, U.S.P grade, micronized) was added to the HA-VS solution.

### Example 33

### Preparation of HA-VS-PEG-(SH)₂ Gel Slurry Containing Triamcinolone Acetonide: Single Extrusion

The triamcinolone acetonide-containing HA-VS/PEG-(SH)₂ gel was physically broken into pieces using a glass rod. The gel was transferred to a sterile 60 mL syringe that was capped with a syringe cap. 40 mL 0.9% sterile NaCl was added to the gel. The plunger was inserted into the syringe barrel and the syringe was inverted. The syringe cap was opened to release any pressure and was then closed. The syringe was inverted several time to ensure good mixing of the saline and the gel pieces. The gel was allowed to swell overnight (at least 16 hrs).

A 23 mm diameter disc of a polyester mesh (McMaster Carr, Cat #9218T13, Mesh Size: 20.3×20.3, Square/Rectangle Size: 0.0331″, Micron Rating: 840 Microns, Percentage of Open Area: 46, Thread Diameter: 0.0157″) was cut out using a 23 mm leather punch. The disc was inserted into a 25 mm syringe filter holder (Cole Palmer, Cat #EW-29550-42) and the filter holder was closed. The filter holder that contained the mesh was autoclaved. The syringe cap of the syringe was removed and the syringe filter containing the mesh was attached to the syringe. The gel was extruded through the mesh into a sterile 50 mL centrifuge tube. The centrifuge tube was capped with a screw top lid. The above steps were performed in a biohood.

1. A method for stabilizing a hyaluronic acid-based polymer composition against a reduction in viscosity over time, the method comprising
incorporating into an aqueous composition comprising a hyaluronic acid polymer and a corticosteroid, a stabilizing amount of a stabilizing additive selected from the group consisting of sugars, sugar alcohols, polyols and polyol-containing surfactants, to thereby provide a composition that exhibits a reduction in viscosity of no more than about 50% in comparison to its initial value when stored at 60° C. for a period of 6 days.

2. A method for diminishing a reduction in viscosity over time of a hyaluronic acid-based polymer composition comprising a corticosteroid, the method comprising
incorporating into an aqueous composition comprising a hyaluronic acid polymer and a corticosteroid, a stabilizing amount of a stabilizing additive selected from the group consisting of sugars, sugar alcohols, polyols and polyol-containing surfactants, to thereby provide a composition having a stabilization ratio of greater than 1.2,
wherein the stabilization ratio is the ratio of the percent retention of viscosity for the hyaluronic acid polymer-corticosteroid composition comprising the stabilizing additive to the percent retention of viscosity of the hyaluronic acid polymer-corticosteroid composition absent the stabilizing additive, when measured at a certain time point and under a defined set of storage conditions.

3. The method of claim 1, wherein the incorporating step comprises incorporating the stabilizing additive into an aqueous composition comprising the hyaluronic acid polymer and corticosteroid, and mixing.

4. The method of claim 1, wherein the hyaluronic acid polymer is selected from the group consisting of unmodified hyaluronic acid which may or may not be crosslinked, chemically-modified hyaluronic acid which may or may not be crosslinked, and combinations thereof.

5. The method of claim 1, wherein the aqueous composition is a solution.

6. The method of claim 5, wherein the solution comprises unmodified hyaluronic acid.

7. The method of claim 1, wherein the aqueous composition comprises a hyaluronic acid polymer-based gel.

8. The method of claim 7, further comprising unmodified hyaluronic acid in solution.

9. The method of claim 7, wherein the hyaluronic acid gel is hyaluronic acid having 10% or less of its hydroxyl groups derivatized by reaction with divinyl sulfone, which is crosslinked with a thiol crosslinker having two or more thiol groups.

10. The method of claim 9, wherein the degree of conversion of the hydroxyl groups of the hyaluronic acid to 2-(vinylsulfonyl)ethoxy groups is selected from 1%, 2%, 3%, 4%, 5%, 6%, 7%, 8%, 9%, and 10%.

11. The method of claim 8, wherein the final hyaluronic acid content in the gel ranges from about 0.05 to 5 percent (0.5 mg/mL to 50 mg/mL).

12. The method of claim 8, wherein the relative amounts (weight ratios) of hyaluronic acid to hydrogel particles in the composition is in a range from about 10:1.

13. The method of claim 1, wherein the stabilizing additive comprises one or more hydroxyl lower alkyl groups.

14. The method of claim 1, wherein the stabilizing additive is selected from the group consisting of sucrose, mannitol, sorbitol, N-acetylglucosamine, dextran-40, polyethylene glycol (PEG), carboxymethylcellulose, TRITON-X-100, glycerol, trehalose, dextrose, maltose and lactose.

15. The method of claim 14, wherein the stabilizing additive is sucrose.

16. The method of claim 1, wherein the amount of stabilizing additive incorporated into the composition ranges from about 0.25 weight % to about 20 weight %.

17. The method of claim 16, wherein the amount of stabilizing additive incorporated into the composition ranges from about 0.5 weight % to about 10 weight %.

18. The method of claim 2, where the incorporating step is effective to provide a composition having a stabilization ratio of greater than 1.5.

19. The method of claim 2, wherein the stabilizing ratio is measured at day 6 upon storage of the composition at 60° C.

20. The method of claim 2, wherein the stabilizing ratio is measured at a time point selected from day 1, day 3, day 5, day 6, day 7, day 10, day 14, day 21, 1 month, 2 months, 3 months, or 6 months, a storage temperature selected from 25° C., 40° C., 45° C., 55° C., 60° C., 80° C., and at a relative humidity in a range from 30% to 75%.

21. The method of claim 1, wherein the corticosteroid is selected from the group consisting of hydrocortisone, hydrocortisone acetate, cortisone acetate, tixocortol pivalate, prednisolone, methylprednisolone, prednisone, triamcinolone, triamcinolone acetonide, triamcinolone benetonide, triamcinolone furetonide, triamcinolone hexacetonide, triamcinolone diacetate, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, beclomethasone dipropionate monohydrate, flunisolide, fluticasone propionate, mometasone furoate monohydrate, and fluticasone furoate.

22. The method of claim 1, where the corticosteroid is triamcinolone acetonide or a pharmaceutically acceptable salt form thereof.

23. The method of claim 22, where the corticosteroid is triamcinolone acetonide.

24. The method of claim 21, wherein the corticosteroid is selected from the group consisting of triamcinolone, triamcinolone acetonide, triamcinolone benetonide, triamcinolone furetonide, triamcinolone hexacetonide, and triamcinolone diacetate.

25. The method of claim 1, wherein the composition comprises from about 0.01% by weight to about 20% by weight corticosteroid.

26. An aqueous composition comprising a hyaluronic acid polymer, a corticosteroid, and a stabilizing additive selected from the group consisting of sugars, sugar alcohols, polyols and polyol-containing surfactants in an amount effective to provide a composition that exhibits a reduction in viscosity of no more than about 50% in comparison to its initial value when stored at 60° C. for a period of 6 days.

27. An aqueous composition comprising a hyaluronic acid polymer, a corticosteroid, and a stabilizing additive selected from the group consisting of sugars, sugar alcohols, polyols and polyol-containing surfactants in an amount effective to provide a composition having a stabilization ratio of greater than 1.2, wherein the stabilizing ratio is the ratio of the percent retention of viscosity for the hyaluronic acid polymer-corticosteroid hydrogel composition comprising the stabilizing additive to the percent retention of viscosity of the hyaluronic acid polymer-corticosteroid hydrogel composition absent the stabilizing additive, when measured at a certain time point and under a defined set of storage conditions.
